(19) United States
(12) Patent Application Publication
(10) Pub. No.: US 2004/0126879 A1
(43) Pub. Date: Jul. 1, 2004

(54) Heart derived cells for cardiac repair

(75) Inventors: Michael D. Schneider, Houston, TX (US); Hidemasa Oh, Houston, TX (US); Mark Entman, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX

(21) Appl. No.: 10/651,548

(22) Filed: Aug. 29, 2003

Related U.S. Application Data
(60) Provisional application No. 60/406,877, filed on Aug. 29, 2002.
(60) Provisional application No. 60/484,612, filed on Jul. 2, 2003.

(51) Int. Cl. C12N 5/08

(52) U.S. Cl. 435/372; 435/366; 424/93.7

(57) ABSTRACT

The present invention is drawn to compositions and methods of using the same to cardiovascular disease. The compositions of the present invention are cardiac stem cells that are c-kit^{neg}/CD31⁺/CD38⁺ and express telomerase reverse transcriptase.

### CROSS-REFERENCE TO RELATED APPLICATIONS

The present invention claims priority to U.S. Provisional Application No. 60/406,877 filed on Aug. 29, 2002 and 60/484,612 filed Jul. 2, 2003 which are incorporated herein by reference in their entirety.

### STATEMENT REGARDING FEDERALLY SPONSORED RESEARCH OR DEVELOPMENT

This invention was made with government support under NHLBI Grant Nos. R01HL47567, R01HL60270, and P01HL499536 awarded by the National Institutes of Health. The United States Government may have certain rights in the invention.

### TECHNICAL FIELD

The present invention relates generally to the field of cardiology. More particularly, the present invention relates to compositions of cardiac stem cells and methods of using the cardiac stem cells to treat cardiovascular disease.

### BACKGROUND OF THE INVENTION

Cardiovascular disease involves diseases or disorders associated with the cardiovascular system. Such disease and disorders include those of the pericardium, heart valves, myocardium, blood vessels, and veins. Of all of the diseases and disorders of the cardiovascular system, coronary artery disease (CAD) is the leading cause of most deaths in the United States. CAD can result from a variety of causes all of which results in reduce blood flow to the myocardium.

A. Myocardial Infarction

Myocardial infarction (MI) is a life-threatening event and may cause cardiac sudden death or heart failure. Despite considerable advances in the diagnosis and treatment of heart disease, cardiac dysfunction after MI is still the major cardiovascular disorder that is increasing in incidence, prevalence, and overall mortality (Eriksson et al., 1995). After acute myocardial infarction, the damaged cardiomyocytes are gradually replaced by fibroid nonfunctional tissue. Ventricular remodeling results in wall thinning and loss of regional contractile function. The ventricular dysfunction is primarily due to a massive loss of cardiomyocytes. It is widely accepted that adult cardiomyocytes have little regenerative capability.

Therefore, the loss of cardiac myocytes after MI is irreversible. Each year more than half million Americans die of heart failure. The relative shortage of donor hearts forces researchers and clinicians to establish new approaches for treatment of cardiac dysfunction in MI and heart failure patients.

B. Cell Transplantation

Recently, cell transplantation has emerged as a potential novel approach for regeneration of damaged myocardium. Transplantation of xenogeneic, allogeneic, and autologous cardiomyocytes, skeletal muscle cells, and smooth muscle cells in normal and injured myocardium has been reported in different species. Several studies have demonstrated the feasibility of engrafting exogenous cells into host myocardium, including fetal cardiomyocytes (Soonpaa et al., 1994), cardiomyocytes derived from artial tumor (ATI) (Koh et al., 1993), satellite cells (Chiu et al., 1995), or bone marrow cells (Tomita et al., 1999). These engrafted cells have been histologically identified in normal myocardium up to 4 months after transplantation (Koh et al., 1993). Cells transplanted close to native cardiomyocytes could form intercalated disks. Gap junctions have been found between the engrafted fetal cardiomyocytes and the host myocardium. (Soonpaa et al., 1994), thereby raising the possibility of an electrical contraction coupling between transplanted cells and the host tissue. Recently, cell transplantation has been extended into ischemically damaged myocardium in rats with coronary artery occlusion (Scorsin et al., 1996; 2000), or in cryoinjured rats (Li et al., 1996) and dogs (Chiu et al., 1995). More recently, Li and his coworkers (Li et al., 2000) showed that autologous porcine heart cell transplantation improved regional perfusion and global ventricular function after a myocardial infarction.

Over the last two decades, the morbidity and mortality of heart failure has markedly increased (Tavazzi, 1998). Therefore, finding an effective therapeutic method is one of the greatest challenges in public health for this century. Although there are several alternative ways for treatment of heart failure, such as coronary artery bypass grafting and whole-heart transplantation, myocardial fibrosis and organ shortage, along with strict eligibility criteria, mandate the search for new approaches to treat the disease. Cell transplantation has emerged as a method to increase the number of contractile myocytes available for the repair of damaged hearts.

Thus, it is necessary to develop alternatives to the cells presently used in transplantation techniques. In light of this need, the present invention is the first to use cardiac stem cells that are CD31⁺, CD38⁺ and c-kit^{neg} to treat damaged myocardium.

### BRIEF SUMMARY OF THE INVENTION

The present invention is directed to a process for isolation of cardiac stem cells and a composition comprising cardiac stem cells. It is envisioned that the cardiac stem cell comprises at least the following characteristics: CD31⁺, CD38⁺ and c-kit^{neg}. In preferred embodiments, the cardiac stem cell is also CD45^{neg}, and/or CD34^{neg}. Thus, the invention is a process for the identification, isolation, and clonal growth of cardiac stem cells. In further embodiments, the present invention comprises methods of administering to a subject isolated cardiac stem cells or a pharmaceutical composition comprising cardiac stem cells to treat cardiovascular disease.

An embodiment of the present invention comprises an isolated mammalian c-kit^{neg}/CD31⁺/CD38⁺ cardiomyocyte stem cell which expresses telomerase reverse transcriptase. Yet further, the cardiac stem cell is also CD45^{neg}, and/or CD34^{neg}. The cell is derived from bone marrow, umbilical cord blood, umbilical tissue, left atrial appendage, cardiac tissue, circulating endothelial progenitor cells, cardiac fibroblasts, adipose tissue or skin tissue. It is envisioned that preferred cardiac stem cells of the present invention possess phenotypic characteristics such as, spontaneous cell beating. The phenotypic characteristic of spontaneous beating may be present at the time the cell is isolated or the cell may acquire or develop this phenotype in time or due to agents or compounds that are added to the cell to force this phenotypic development. In further embodiments, the cardiac stem cells may also be capable of differentiating into cardiac muscle or vascular cells.

Still further, the stem cell expresses an adhesion protein. Exemplary adhesion proteins are selected from the group consisting of annexin A1 (Anxa1), nephronectin (Npnt), nidogen 2 (Nid2), pentaxin 3 (Ptx3), transmembrane 4 superfamily member 6 (Tm4sf6), and vascular cell adhesion molecule 1 (Vcam1). The cell can also express macrophage colony stimulating factor 1. Still further, the cell can also express a receptor, for example, but not limited to fibroblast growth factor receptor 1 (Fgfr1), cytokine receptor-like factor 1 (Crlf1), interleukin 4 receptor alpha (Il4ra), platelet derived growth factor receptor alpha polypeptide (Pdgfra), and tumor necrosis factor receptor superfamily member 6 (Tnfrsf6).

Another embodiment of the present invention is an isolated c-kit negative cardiac derived stem cell. The cell also expresses CD31/PECAM-1, CD38, or telomerase reverse transcriptase. The cardiac derived stem cell is isolated from cardiac tissue. Still further the cell is isolated from a non-myocyte fraction, and is capable of differentiating into cardiac muscle or vascular cells.

A further embodiment of the present invention is a pharmaceutical composition comprising a therapeutically effective amount of the isolated stem cells admixed with a pharmaceutically acceptable carrier. More specifically, the pharmaceutical composition further comprises a transcription factor for cardiac development, for example, Nkx2.5, or a factor that enhances the activity of the Wnt/β-catenin signaling pathway, or a factor that enhances the activity of BMP (bone morphogenic protein).

A specific embodiment of the present invention is a method of treating a subject suffering from a cardiovascular disease comprising the step of administering to the subject cardiac stem cells, wherein the stem cells are c-kit^{neg}/CD31⁺/CD38⁺ and express telomerase reverse transcriptase. The cells are autologous, heterologous, or homologous. More specially, the cells differentiate into at least one cardiac cell type selected from the group consisting of myocytes, endothelial cells, vascular smooth muscle cells, and fibroblasts.

The cardiovascular disease is selected from the group consisting of coronary artery disease, myocardial infarction, ischemic heart disease and heart failure. The cells are administered via a parenteral route, for example, intravenously, or via direct injection into the heart of the subject. The cells can also be combined with a matrix and the matrix is implanted into the subject.

Another embodiment is a method of treating a subject suffering from an infarcted myocardium comprising the step of administering to the subject an effective amount of stem cells that are C-kit^{neg}/CD31⁺/CD38⁺ and express telomerase reverse transcriptase, wherein the amount repairs the infarcted myocardium. The repairs comprise regeneration of cardiomyocytes.

Still further, another embodiment is a method of targeting injured myocardium comprising the step of administering to the subject stem cells that are c-kit^{neg}/CD31⁺/CD38⁺ and express telomerase reverse transcriptase, wherein the cells migrate and attach to the injured myocardium.

Another embodiment is method of repairing an injured myocardium comprising the step of administering to a subject an effective amount of stem cells that are c-kit^{neg}/CD31⁺/CD38⁺ and express telomerase reverse transcriptase, wherein the amount is effective in repairing the injured myocardium. Repairing the myocardium comprises at least partially restoring structural integrity or functional integrity to the injured myocardium.

Still further, another embodiment is a method of repairing injured coronary vessels comprising the step of administering to a subject an effective amount of stem cells that are c-kit g/CD31⁺/CD38⁺ and express telomerase reverse transcriptase, wherein the amount is effective in regenerating vascular cells to repair the vessels.

A further embodiment is a method of generating myocytes comprising the steps of: obtaining cardiac stem cells that are c-kit^{neg}/CD31⁺/CD38⁺ and express telomerase reverse transcriptase; and differentiating the stem cells to generate myocytes in vitro. The stem cells are further differentiated by the addition of a transcription factor for cardiac development, for example, Nkx2.5.

Another embodiment is a method of treating damaged myocardium in a subject comprising the steps of: obtaining autologous stem cells that are c-kit^{neg}/CD31⁺/CD38⁺ and express telomerase reverse transcriptase from the subject; proliferating the stem cells in vitro; and administering intravenously to the subject the stem cells, wherein the stem cells migrate to the damaged myocardium. The cells can be derived from a tissue biopsy from the subject.

Another embodiment of the present invention comprises a method of treating heart failure in a subject comprising the step of administering to the subject an effective amount of cardiac stem cells that are c-kit^{neg}/CD31⁺/CD38⁺ and express telomerase reverse transcriptase, wherein the amount is effective in at least partially restoring cardiac function. The heart failure may comprise the loss of cardiomyocytes, which may be a result of apoptotic mechanisms. More specifically, the administration of the stem cells at least partially counteracts the loss of cardiomyocytes or restores the cardiomyocytes.

A further embodiment is a method of modulating the loss of cardiomyocytes in a subject comprising the step of administering to the subject an effective amount of cardiac stem cells that are c-kit^{neg}/CD31⁺/CD38⁺ and express telomerase reverse transcriptase, wherein the amount is effective in at least partially restoring cardiomyocytes. A loss of cardiomyocytes can be related to heart failure and apoptosis. It is envisioned that administration of the cells to a subject that has suffered a loss of cardiomyocytes may treat and/or prevent heart failure in the subject.

Still further, another embodiment is a kit comprising a pharmaceutical composition of the stem cells of the present invention to treat a cardiovascular disease.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

The patent or application file contains at least one drawing executed in color. Copies of this patent or patent application publication with color drawing(s) will be provided by the Office upon request and payment of the necessary fee.

FIG. 1A-FIG. 1C show the isolation Sca-1+ cells from adult mouse myocardium. Flow cytometry was used to analyze the isolated cells for Sca-1 (FIG. 1B (total heart) and FIG. 1C (myocyte-depleted)). IgG2a+2b−FITC was used as the control (FIG. 1A).

FIG. 2A and FIG. 2B show immunostaining of adult mouse myocardium for Sca-1, laminin (FIG. 2A), and CD31 (FIG. 2B). Yellow-orange in the merged images denotes co-localization. Representative cells are highlighted in white, and shown at higher magnification in the insets. The bar represents 15 μm.

FIG. 3A-FIG. 3F show cells that were labeled with Sca-1 in tandem with the indicated markers and measured using flow cytometry. FIG. 3A shows cells that were labeled with IgG2a+2b−FITC, which was used as the control. FIG. 3B shows cells that were labeled with Sca-1-PE-Lin-FITC. FIG. 3C shows cells that were labeled with c-kit-Sca-1-FITC. FIG. 3D shows cells that were labeled with Sca-1-PE-CD45-FITC. FIG. 3E shows the values from the flow cytometry data in a bar graph, which denotes cell prevalence in the myocyte-depleted population. FIG. 3F shows the values from flow cytometry data in a bar graph for bone marrow cell±collagenase.

FIG. 4A-FIG. 4D show cardiac SP cells that were identified with Hoechst 33342 (FIG. 4A). Labeling with Sca-1 (FIG. 4B) versus c-kit (FIG. 4C) and CD45 (FIG. 4D) is shown in the contour plots.

FIG. 5 shows enrichment for SP cells in the cardiac Sca-1+ population.

FIG. 6A-FIG. 6F show the purity of cardiac Sca-1+ and Sca-1− cells after magnetic enrichment. FIG. 6A and FIG. 6B show analysis by flow cytometry. FIG. 6C-FIG. 6F show corroborative immunostaining for Sca-1. The Bar is equivalent to 5 μm.

FIG. 7 shows telomerase activity that was detected in cardiac Sca-1+ cells, but not Sca-1− cells. Neonatal mouse heart was used as a positive control (neonate); numbers above each lane indicate the amount of adult lysate, relative to the control.

FIG. 8A and FIG. 8B show RT-PCR analysis of cardiac Sca-1+ and Sca-1− cells. Adult mouse heart was used for comparison.

FIG. 9A-FIG. 9D show in vitro differentiation of cardiac Sca-1+ cells induced by 5-aza. FIG. 9A and FIG. 9B show in vitro differentiation after 5 days in the presence (FIG. 9A) or absence of 5-aza (FIG. 9B). FIG. 9C and FIG. 9D show in vitro differentiation after 14 days in the presence (FIG. 9C) or absence of 5-aza (FIG. 9D). The bar is equivalent to 20 μm.

FIG. 10A-FIG. 10D show induction of sarcomeric α-actin (FIG. 10A and FIG. 10C) and cardiac troponin-I (c TN I) (FIG. 10B and FIG. 10D) using 5-aza, shown by immunostaining 4 wk after treatment. Differentiated cells (red) are found in the monolayer. DAPI is blue. The bar is equivalent to 10 μm.

FIG. 11 shows the induction of Nkx-2.5, ALK3, and cardiac myosin heavy chain genes using 5-aza, show by RT-PCR.

FIG. 12A and FIG. 12B show excision of floxed Bmpr1a allele by adenoviral delivery of Cre. FIG. 12A shows a cartoon of the floxed and null Bmpr1a alleles at exon 2. FIG. 12B shows by PCR excision of exon 2 after delivery of Cre.

FIG. 13A-FIG. 13F show the analysis of cardiac Sca-1⁺ cells isolated from Bmpr1a^{F/-} mice that were subjected to viral gene transfer and differentiated with 5-aza. Analysis was preformed using quantitative RT-PCR for the following markers BMP-2 (FIG. 13A), BMP-4 (FIG. 13B), Nkx-2.5 (FIG. 13C), Tbx-5 (FIG. 13D), MEF-2c (FIG. 13E), and α-MHC (FIG. 13F).

FIG. 14A-FIG. 14bb show that cardiac Sca-1⁺ cells home to injured myocardium and differentiate in situ. FIG. 14A-FIG. 14F show engraftment in the anterolateral infarct border zone, 2 wk after intravenous injection. FIG. 14F-FIG. 14H show higher power images of donor-derived myocytes identified by sarcomeric α-actin and cardiac troponin-I. No engraftment occurred in the interventricular septum (IVS, FIG. 14I-FIG. 14J) or posterior wall (FIG. 14K-FIG. 14L). FIG. 14M-FIG. 14X show that grafted cells express the smooth muscle and endothelial markers. For SM-MHC, a merged image is shown, and donor-donor-derived smooth muscle cells are shown by the arrow heads. FIG. 14Y-FIG. 14bb show donor cells that were accumulated in the spleen, but not liver, lung, or kidney.

FIG. 15 shows RT-PCR analysis showing lack of Cre expression in newly isolated cardiac Sca-1+ cells from αMHC-Cre mice.

FIG. 16 shows a Western blot showing expression of neo in R26R mice.

FIG. 17A-FIG. 17D show homing of dye-labeled cardiac Sca-1+ cells. FIGS. 17A and 17B show cells after 24 hr of engraftment and FIG. 17C and FIG. 17D show cells after 2 weeks of engraftment. The bar represents 20 μm.

FIG. 18A-FIG. 18D show that Neo (FITC; yellow in merged image) was ubiquitously expressed in adult ventricular myocardium of R26R mice (FIG. 18C and FIG. 18D). No staining was seen in C57BL/6 mice (FIG. 18A and FIG. 18B), which was the negative control. Cardiomyocytes were identified by sarcomeric α-actin (Texas Red) and nuclei by DAPI. The bar represents 20 μm.

FIG. 19A-FIG. 19D show samples from animals that were analyzed by confocal microscopy 2 weeks after ischemia/reperfusion injury and infusion of Sca1+ cells. FIG. 19A show that Neo was ubiquitously expressed in adult ventricular myocardium of R26R mice (ntg, non-transgenic). Grafted Sca-1+ cells from αMHC-Cre mice activate the myocyte-specific Cre gene and recombination of R26R. Cre protein (green) was localized to nuclei (DAPI) as shown in FIG. 19B. Unfused, donor-derived cells express neither neo nor LacZ as shown in FIG. 19B. Fusion with host R26R myocardium typically results in LacZ+ muscle cells (red; FIG. 19, circled). Fused cells without recombination as shown in FIG. 19D (circled) were detected rarely. The bar represents 20 μm.

FIG. 20 shows donor-derived myocytes with (Cre+ LacZ+neo−; Cre+LacZ− neo+) and without (Cre+LacZ− neo−) fusion after grafting. BZ, infarct border zone in anterolateral (A, L) myocardium; NI, non-infarcted control regions (P, posterior wall; R, right ventricle; interventricular septum).

FIG. 21A-FIG. 21B show delineation of Cre+ cells by laminin. FIG. 21A shows myocytes derived from non-transgenic mice (ntg). FIG. 21B shows myocytes derived from R26R mice. The bar represents 20 μm.

FIG. 22A-FIG. 22D show all Cre+ cells co-expressed α-actin (FIG. 22A), c Tn I (FIG. 22B), and Cx43 (FIG. 22C). Mitotic phosphorylation of histone H3 two wk after grafting was seen almost exclusively in donor-derived Cre+ myocytes (FIG. 22D). In FIG. 22D, the arrow shows Cre+ phospho-H3+ cardiomyocyte nuclei (blue-green in merged image). The bar represents 20 μm.

### DETAILED DESCRIPTION OF THE INVENTION

The present invention relates to compositions and methods of using the same to treat cardiovascular diseases.

As used herein, the use of the word “a” or “an” when used in conjunction with the term “comprising” in the claims and/or the specification may mean “one,” but it is also consistent with the meaning of “one or more,” “at least one,” and “one or more than one.”

As used herein, the term “allogeneic” refers to cells being genetically different, but deriving from the same species.

As used herein, the term “assemble” refers to the assembly of differentiated stem cells into functional structures i.e., myocardium and/or myocardial cells, coronary arteries, arterioles, and capillaries etc. This assembly provides functionality to the differentiated myocardium and/or myocardial cells, coronary arteries, arterioles and capillaries.

As used herein, the term “autologous” refers to tissue, cells or stem cells that are derived from the same subject's body.

As used herein, the term “cardiac stem cell” refers to stem cells that are capable of differentiating into a cardiomyocyte.

As used herein, the term “cardiovascular disease or disorder” refers to disease and disorders related to the cardiovascular or circulatory system. Cardiovascular disease and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skill in the art recognizes that cardiovascular diseases and/or disorders can result from congenital defects, genetic defects, environmental influences (i.e., dietary influences, lifestyle, stress, etc.), and other defects or influences.

As used herein, the term “cardiomyocyte” refers to any cell in the cardiac myocyte lineage that shows at least one phenotypic characteristic of a cardiac muscle cell. Such phenotypic characteristics can include expression of cardiac proteins, such as cardiac sarcomeric or myofibrillar proteins or atrial natriuretic factor, or electrophysiological characteristics. As used herein, the term “cardiomyocyte” and “myocyte” are interchangeable.

As used herein, the term “cell surface marker” refers to a protein, glycoprotein or other molecule expressed on the surface of a cell, which serves to identify the cell. A cell surface marker can generally be detected by conventional methods, for example, but not limited to immunohistochemistry, fluorescence activated cell sorting (FACS), or an enzymatic analysis.

As used herein, the term “c-kit” refers to a cell surface marker. c-Kit is also known as CD117 or stem-cell factor receptor (SCF). Typically, c-kit is expressed on hematopoietic progenitor cells. Yet further, those of skill in the art realize that c-kit is also related to the immunoglobulin family and the tyrosine kinase family.

As used herein, the term “c-kit negative” or “c-kit^{neg}” refers to a cell in which a c-kit surface marker or a structural or functional equivalent of c-kit is absent. Thus, one of skill in the art realizes that a “c-kit negative” cell is a cell that is not a hematopoietic cell.

As used herein, the term “coronary artery disease” (CAD) refers to a type of cardiovascular disease. CAD is caused by gradual blockage of the coronary arteries. One of skill in the art realizes that in coronary artery disease, atherosclerosis (commonly referred to as “hardening of the arteries”) causes thick patches of fatty tissue to form on the inside of the walls of the coronary arteries. These patches are called plaque. As the plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack.

As used herein, the term “damaged myocardium” refers to myocardial cells which have been exposed to ischemic conditions. These ischemic conditions may be caused by a myocardial infarction, or other cardiovascular disease or related complaint. The lack of oxygen causes the death of the cells in the surrounding area, leaving an infarct, which will eventually scar.

As used herein, the term “heart failure” refers to the loss of cardiomyocytes such that progressive cardiomyocyte loss over time leads to the development of a pathophysiological state whereby the heart is unable to pump blood at a rate commensurate with the requirements of the metabolizing tissues or can do so only from an elevated filling pressure. The cardiomyocyte loss leading to heart failure may be caused by apoptotic mechanisms.

As used herein, the term “heterologous” refers to tissue, cells or stem cells that are derived from the different species.

As used herein, the term “homologous” refers to tissue, cells or stem cells that are derived from the same species.

As used herein, “home” refers to the attraction and mobilization of stem cells toward damaged myocardium and/or myocardial cells.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “ischemic heart disease” refers to a lack of oxygen due to inadequate perfusion or blood supply. Ischemic heart disease is a condition having diverse etiologies. One specific etiology of ischemic heart disease is the consequence of atherosclerosis of the coronary arteries.

As used herein, the term “myocardium” refers to the muscle of the heart.

As used herein, the term “myocyte” refers to a muscle cell, i.e., cardiac muscle. As used herein the terms myocyte and cardiomyocyte are interchangeable.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

As used herein, the term “progenitor cell” refers to a cell that is an undifferentiated cell that is capable of differentiating. One of skill in the art realizes that a progenitor cell is an ancestor cell to progeny descendant cells.

As used herein, the term “stem cell” refers to an “undifferentiated” cell capable of proliferation, self-maintenance, production of a differentiated cell or regeneration of a stem cell may be tissue. In preferred embodiments of the present invention, a stem cell is capable of differentiating into a differentiated myocardial cell, such as a cardiomyocyte.

As used herein, the term “subject” may encompass any vertebrate including but not limited to humans, mammals, reptiles, amphibians and fish. However, advantageously, the subject is a mammal such as a human, or other mammals such as a domesticated mammal, i.e., dog, cat, horse, and the like, or production mammal, i.e., cow, sheep, pig, and the like

The term “therapeutically effective amount” as used herein refers to an amount that results in an improvement or remediation of the symptoms of the disease or condition.

The term “treating” and “treatment” as used herein refers to administering to a subject a therapeutically effective amount of a the composition so that the subject has an improvement in the disease. The improvement is any improvement or remediation of the symptoms. The improvement is an observable or measurable improvement. Thus, one of skill in the art realizes that a treatment may improve the disease condition, but may not be a complete cure for the disease.

As used herein, the term “xenogeneic” refers to cells that are derived from different species.

I. Cardiac Stem Cells

An embodiment of the present invention is isolated cardiac stem cells. Thus, the instant invention is a process for isolation of cardiac stem cells and a composition comprising cardiac stem cells. It is envisioned that the cardiac stem cell comprises at least the following characteristics CD31⁺, CD38⁺, and C-kit^{neg}. The stem cell also expresses telomerase reverse transcriptase. Still further, the stem cells are also CD45^{neg}, and/or CD34^{neg}. Thus, the invention is a process for the identification, isolation, and clonal growth of cardiac stem cells.

A. Isolation of Stem Cells

It is envisioned that stem cells that are capable of differentiating into a cardiomyocyte cell have “cardiomyocyte potential”. Cardiomyocyte potential refers to the ability to give rise to progeny that can differentiate into a cardiomyocyte under specific conditions. Examples of stem cells with cardiomyocyte potential include pluripotent cells, progenitor cells (i.e., circulating endothelial progenitor cells or hemangioblasts), stem cells (i.e., hematopoietic stem cells, embryonic stem cells, or fibroblasts (i.e., muscle fibroblast, cardiac fibroblast, etc.). Stem cells can be isolated from embryonic or nonembryonic donors. The tissues from which the stem cells can be isolated include, for example, but are not limited to the bone marrow, the spleen, the liver, peripheral blood, umbilical cord tissue, umbilical cord blood, adipose tissue or skin. The stem cells are isolated using standard techniques well known and used in the art, for example, but not limited to those described in U.S. Patent Application No. US20020142457, U.S. Patent Application No. US20030082153 and International Patent Application No. WO011011, which are incorporated herein by reference. The donor tissue or sample can be isolated from a vertebrate, more particularly a mammal, for example, human, dog, cat, monkey, mouse, rat, bird, etc. More preferably the mammal is an adult mammal. In preferred embodiments, the mammal is a human. The tissue and/or sample can include the entire tissue or sample, a portion of a tissue or sample, or biopsy sample.

In a specific embodiment, it is envisioned that cardiac stem cells are isolated from heart tissue, thus cardiac-derived stem cells. The heart tissue can be isolated from a vertebrate, more particularly a mammal. More preferably the mammal is an adult mammal. The tissue can include the entire heart, a portion of a heart, or biopsy sample.

Any method of isolating cardiac stem cells is acceptable, including affinity-based interactions, affinity panning, or flow cytometry. In preferred embodiments, flow cytometry is used to determine the fraction of cells that are the “cardiac stem cell” fraction of the present invention. Flow cytometry involves the separation of cells or other particles in a liquid sample. Generally, the purpose of flow cytometry is to analyze the separated particles for one or more characteristics thereof. The basic steps of flow cytometry involve the direction of a fluid sample through an apparatus such that a liquid stream passes through a sensing region. The particles should pass one at a time by the sensor and are categorized base on size, refraction, light scattering, opacity, roughness, shape, fluorescence, etc.

Not only can cell analysis be performed by flow cytometry, but cell sorting can also be performed. In U.S. Pat. No. 3,826,364, an apparatus is disclosed which physically separates particles, such as functionally different cell types. In this machine, a laser provides illumination which is focused on the stream of particles by a suitable lens or lens system so that there is highly localized scatter from the particles therein. In addition, high intensity source illumination is directed onto the stream of particles for the excitation of fluorescent particles in the stream. Certain particles in the stream may be selectively charged and then separated by deflecting them into designated receptacles. A classic form of this separation is via fluorescent tagged antibodies, which are used to mark one or more cell types for separation. Antibodies that may be marked and used in the present invention include anti-Sca-1, anti-c-kit, anti-CD4, anti-CD8, anti-B220, anti-Gr-1, anti-Mac-1, anti-TER119, anti-CD-45, anti-CD34, and Flt-1, anti-FLk-1, anti-VE-cadherin, anti-vWf (von Willebrand factor), anti-CD-38, and anti-CD31/PECAM-1 (platelet endothelial cell adhesion molecule-1).

In a preferred embodiment, the isolated cardiac stem cells of the present invention do not express hematopoietic stem cell markers (CD45, CD34 and c-kit), blood cell lineage markers and endothelial progenitor cell markers (CD45, CD34, Flk-1, and Flt-1). Thus, the lack of the expression of these markers, distinguishes the cardiac stem cells of the present invention from hematopoietic stem cells, blood cells, and endothelial stem cells. In fact, the cardiac stem cells of the present invention closely resemble the myogenic “satellite” cells of skeletal muscle, which are CD31⁺, CD38⁺, CD45^{neg}, CD34^{neg}, and c-kit^{neg}.

Preferred cardiac stem cells of the present invention express transcription factors that are necessary for cardiac development. The transcription factors can include one or more, but are not limited to ATA-4, MEF-2C, TEF-1, MLP/CRP1, MLP/CRP2, Tie-2, SRF, or Ang1. Yet further, the cardiac stem cells of the present invention express telomerase reverse transcriptase (TERT). Those of skill in the art are well aware that telomerase activity is also associated with self-renewal.

Still further, the cardiac stem cells of the present invention also express cell markers such as CD-38, and CD31/PECAM-1 (platelet endothelial cell adhesion molecule-1). Other markers that can be used to identify the stem cells of the present invention include, but are not limited to those listed in Table 1 of the present application, which is incorporated herein. Such examples, can include, fibroblast growth factor receptor 1, cytokine receptor-like factor 1, interleukin 4 receptor alpha, platelet derived growth factor receptor alpha polypeptide, tumor necrosis factor receptor superfamily member 6, and macrophage colony stimulating factor 1.

Moreover, those of skill in the art understand that the cells of the present invention can be identified and purified on the basis of markers expressed inside the cell, not just those outside the cell, if a hybrid gene is first put into the cell, encoding a readily assayable marker and controlled transcriptionally by regulatory elements from non-coding DNA sequences of the gene whose expression is to be denoted by the hybrid reporter. Examples of readily assayable markers include, but are not limited to (i) spontaneously fluorescent proteins such as green fluorescent protein, cyan fluorescent protein, yellow fluorescence protein, and red fluorescence protein; or (ii) surface proteins not expressed otherwise expressed by the cell receiving the marker gene. Examples of regulatory elements are promoters and enhancers. Thus, as an illustrative example, one skilled in the art realizes that the TERT promoter can be used to cause fluorescent protein expression in cells that express TERT, as a surrogate marker for TERT itself. Another example relates to utilizing the Tbx-5 promoter in a similar capacity as the TERT promoter. Thus, the Tbx-5 promoter drives the expression of GFP or a similar reporter as a way to determine that the isolated cells possess the properties of the stem cells of the present invention. Other promoters that may be used in a similar fashion include, but are not limited to Bop, Popeye and the SRF 3′UTR.

B. Enhancing Cell Differentiation

Enhancing differentiation of a cell refers to the act of increasing the extent of the acquisition or possession of one or more characteristics or functions which differ from that of the original cell (i.e., cell specialization). This can be detected by screening for a change in the phenotype of the cell (i.e., identifying morphological changes in the cell and/or surface markers on the cell).

Enhancing conversion or differentiation of a stem cell into a cardiomyocyte cell includes the act of increasing the extent of the acquisition or possession of one or more characteristics or functions that are used to identify a cell as a cardiomyocyte. For example, a specific function can include spontaneous beating, however, the present invention is not limited to this function of spontaneous beating. Other functional properties include, but are not limited to cardiac differentiation in tissue culture in response to 5′azacytidine, to other inhibitors of DNA methylase or DNA methylation, or cardiac differentiation in tissue culture that is dependent on bone morphogenetic proteins (BMPs) or their receptors regardless of the instigating signal. As used in the present invention, the term differentiation and conversion may be interchangeable. The conversion of a stem cell into a cardiomyocyte cell includes enhancing factors that are known to be required to convert cells into to cardiomyocytes for example, BMP or Wnt. Such factors and techniques to convert stem cells into cardiomyocytes are described in U.S. Provisional Application 60/464,292 filed on Apr. 21, 2003, which is incorporated herein by reference.

Methods for inducing cardiomyocytes from the stem cells having the potential to differentiate into cardiomyocytes include, but are not limited to the following: induction of differentiation by the treatment with a DNA-demethylating agent, induction of differentiation using a factor which is expressed in the cardiogenesis region of a fetus or a factor which controls differentiation into cardiomyocytes in the cardiogenesis stage of a fetus, and induction of differentiation using a culture supernatant of the cells having the potential to differentiate into cardiomyocytes or cardiomyocytes differentiated from the cells. Cardiomyocytes can be induced from the cells having the potential to differentiate into cardiomyocytes using such a method alone or in combination. Also, according to these methods, even mesenchymal cells which originally do not have the potential to differentiate into cardiomyocytes can be differentiated into cells having the potential to differentiate into cardiomyocytes, and cardiomyocytes can be induced from the original mesenchymal cell population.

Treatment with any DNA-demethylating agent can be used, so long as it is a compound which causes demethylation of DNA. Suitable DNA-demethylating agents include demethylase that is an enzyme which specifically removes the methylation of the cytosine residue in the GpC sequence in a chromosomal DNA, 5-azacytidine (5-aza-C) and DMSO (dimethyl sulfoxide).

Examples of the factors which are expressed in the cardiogenesis region of a fetus and the factors which act on differentiation into cardiomyocytes in the cardiogenesis stage of a fetus include cytokines, growth factors, vitamins, adhesion molecules and transcription factors.

Any cytokine and/or growth factor can be used, so long as it stimulates the cardiomyogenic differentiation of the cells having the potential to differentiate into cardiomyocytes in the cardiogenesis stage. Examples include bone morphogenic protein (BMP), macrophage colony stimulating factor, bone morphogenetic proteins (BMPs), insulin-like growth factor 1 (Igf1) or adrenomedullin.

It is also possible to stimulate the cardiomyogenic differentiation of the cells having the potential to differentiate into cardiomyocytes in the cardiogenesis stage using an inhibitor against a cytokine which suppresses the cardiomyogenic differentiation. The cytokines, which suppress the cardiomyogenic differentiation, include fibroblast growth factor-2. The inhibitors against the cytokines which suppress the cardiomyogenic differentiation include substances which inhibit the signal transduction of the cytokines, such as antibodies and low molecular weight compounds which neutralize the cytokine's activities.

It is also envisioned that other repressors of differentiation can also be employed to result in cardiomyogenic differentiation. Such repressors include, but are not limited to DNA methyltransferase (cytosine-5) 1; histone deacetylase 1; hairy/enhancer-of-split related with YRPW motif 1; Smad 7; runt related transcription factor 1 (runx 1); and runt related transcription factor 2 (runx 2).

Any vitamin can be used, so long as it stimulates the cardiomyogenic differentiation of the cells having the potential to differentiate into cardiomyocytes in the cardiogenesis stage.

Any adhesion molecule can be used, so long as it is expressed in the cardiogenesis region in the cardiogenesis stage. Examples include extracellular matrices such as gelatin, laminin, collagen, fibronectin and the like. For example, the cardiomyogenic differentiation of the cells having the potential to differentiate into cardiomyocytes can be stimulated by culturing the cells on a culture dish coated with fibronectin.

Enhancement of cell differentiation can also include forcing expression of cardiac transcription factors. Examples of the transcription factors include a homeobox-type transcription factor, Nkx2.5/Csx, a zinc finger-type transcription factor belonging to the GATA family (GATA4); transcription factors belonging to the myocyte enhance factor-2 (MEF-2) family (MEF-2, MEF-2B, MEF-2C, and MEF-2D); transcription factors belonging to the basic helix loop helix-type transcription factors (DHAND, eHAND); transcription factors belonging to the family of TEA-DNA binding-type transcription factors (TEF-1, TEF-3 and TEF-5); and transcription factors belonging to the LIM domain-containing members of the cysteine-rich protein (CRP1, CRP2/SmLIM and CRP3/MLP). Other transcription factors include, but are not limited to Csrp3, Pop 3 and Bop.

Still further, cell cycle proteins can be used to enhance cell differentiation of stem cells into cardiac stem cells. Examples include, but are not limited to cyclin-dependent kinases (CDK) and cyclins (i.e., cyclin D2). It is also contemplated that interference of growth suppressors, i.e., cyclin-dependent kinase inhibitors, tumor suppressor p53, etc., can also play a role in cell differentiation

The cardiomyogenic differentiation of the cells having the potential to differentiate into cardiomyocytes can be induced by introducing DNA encoding one or combination of the above-described factors into the cells and expressing the DNA therein using standard molecular biological techniques, for example, the use of expression vectors. Development of expression vectors are well known and used in the art, for example Maniatis et al., 1982. Once the expression vector is generated it can be delivered to the cells via standard transfection protocols, which are known and used in the art. These standard transfection protocols include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

It is also contemplated in the present invention that double-stranded RNA is used as an interference molecule, i.e., RNA interference (RNAi). RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et al., 2000).

In certain embodiments, the present invention utilizes the technique of chemically induced dimerization (CID) to produce a conditionally controlled protein or peptide. In addition to this technique being inducible, it also is reversible, due to the degradation of the labile dimerizing agent or administration of a monomeric competitive inhibitor (U.S. Pat. Nos. 5,869,337; 5,830,462; 5,834,266; and 6,046,047; and US Patent Application No. 20030144204, which are incorporated herein by reference).

It is also possible to induce the cardiomyogenic differentiation of the cells having the potential to differentiate into cardiomyocytes by culturing them using a culture dish coated with an extracellular matrix obtained from spontaneously beating cardiomyocytes, co-culturing with spontaneously beating cardiomyocytes or adding a culture supernatant of spontaneously beating cardiomyocytes.

Furthermore, factors which induces differentiation of cardiomyocytes which are obtained by the method described herein hereinafter referred to as “the cardiomyogenic differentiation-inducing factor”) can also be used in inducing the cardiomyogenic differentiation of the cells having the potential to differentiate into cardiomyocytes. A cardiomyogenic differentiation-inducing factor can be obtained by adding various protease inhibitors to a culture supernatant of spontaneously beating cardiomyocytes, followed by combinations of treatments, such as dialysis, salting-out and chromatography. Genes encoding such cardiomyogenic differentiation-inducing factors can be obtained by determining partial amino acid sequences of these factors using a microsequencer followed by screening a cDNA library prepared from spontaneously beating cells using DNA probes designed based on the determined amino acid sequences (US Patent Application No. US20020142457, which is incorporated herein by reference).

C. Enhancing Cell Survivability

To promote cell survivability, the present invention can utilize known mechanisms to antagonize apoptosis. Apoptosis involves two essential steps. The Bcl-2 family of proteins that consists of different anti- and pro-apoptotic members is important in the “decision” step of apoptosis. In contrast, the “execution” phase of apoptosis is mediated by the activation of caspases. Bcl-2-related proteins act upstream from caspases in the cell death pathway. Over-expression of Bcl-2 suppresses apoptosis induced by a variety of agents both in vitro and in vivo. Based on their differential roles in regulating apoptosis, the Bcl-2-related proteins can be separated into anti-apoptotic (Bcl-2, Bcl-xL, Mcl-1, Bcl-w and Bfl-1/A1) and pro-apoptotic members (Bax, BAD, Bak, Bik, Hrk and BID).

Thus, it is contemplated in the present invention that anti-apoptotic proteins can be administered to the stem cells or cardiomyocytes of the present invention. The anti-apoptotic proteins can be introduced into the cells of the present invention using the standard molecular biological techniques described in the above section, which are incorporated herein. Examples of the anti-apoptotic proteins that can be introduced into the cells include, but are not limited to, Akt, Bcl-2, and survivin. (See US Patent Applications US20030144204, US20030049709, and U.S. Pat. No. 6,509,162, and 6,222,017 which are incorporated herein by reference). Still further, since gene expression appears to be required for cell death, cell death can also be prevented by inhibitors of RNA or protein synthesis (Cohen, 1984; Stanisic et al. 1978; Martin et al., 1988).

It is also contemplated that proteins or factors that are involved in the mitochondrial death pathway of cardiomyocytes can be inhibited in the present invention to increase the life-span of the cardiac stem cells (Crow, 2002, Kubasiak et al., 2002, Bishopric et al., 2001).

D. Purification of Stem Cells

In further embodiments, it may be desirable to purify the cardiac stem cells. Purification techniques are well known to those of skill in the art. Analytical methods particularly suited to the cell purification and preparation of the present invention include affinity chromatography and variations thereof, immunohistochemistry, fluorescence activated cell sorting (FACS), or an enzymatic analysis.

Certain aspects of the present invention concern the purification, and in particular embodiments, the substantial purification, of the cells. The term purified cell as used herein, is intended to refer to a composition, isolatable from other components, wherein the cell is purified to any degree relative to its naturally-obtainable state. A purified cell therefore also refers to a cell, free from the environment in which it may naturally occur.

Various techniques suitable for use in cell purification are well known to those of skill in the art. These include, for example, centrifugation, chromatography and combinations of such and other techniques. As is generally known in the art, it is believed that the order of conducting the various purification steps may be changed, or that certain steps may be omitted, and still result in a suitable method for the preparation of a substantially purified cell or cell fraction.

A relatively pure population or substantially pure population refers to a population of cells comprising at least about 80% cells with cardiomyocyte cell potential. More preferably, the population comprises at least about 90% cells with cardiomyocyte cell potential. Even more preferably, the population comprises at least about 95% cells with cardiomyocyte cell potential. Most preferably, the population comprises at least about 99% cells with cardiomyocyte cell potential.

There is no general requirement that the cell always be provided in the most purified state. Indeed, it is contemplated that less substantially purified products will have utility in certain embodiments. Partial purification may be accomplished by using fewer purification steps in combination, or by utilizing different forms of the same general purification scheme.

In preferred embodiments, affinity chromatography is used to purify the cardiac stem cells of the present invention. Affinity chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.). An example of affinity chromatography that can be utilized in the present invention includes, but is not limited to constructing an affinity column with anti-CD31 and/or anti-CD38 antibodies.

Another preferred embodiment is to utilize fluorescence activated cell sorting (FACS) to purify the cells, as described in U.S. Pat. No. 3,826,364, which is incorporated herein by reference. In this process, fluorescent tagged antibodies are used to mark one or more cell types for separation. Antibodies that may be marked and used in the present invention include anti-Sca-1, anti-c-kit, anti-CD4, anti-CD8, anti-B220, anti-Gr-1, anti-Mac-1, anti-TER119, anti-CD-45, anti-CD34, and Flt-1, anti-FLk-1, anti-VE-cadherin, anti-vWf (von Willebrand factor), anti-CD-38, and anti-CD31/PECAM-1 (platelet endothelial cell adhesion molecule-1).

II. Pharmaceutical Compositions

The present invention provides a pharmaceutical composition comprising cardiac stem cells and a pharmaceutical carrier. The pharmaceutical compositions of the present invention are used to treat cardiovascular diseases, including, but not limited to, coronary heart disease, arteriosclerosis, ischemic heart disease, angina pectoris, myocardial infarction, congestive heart failure and other diseases of the arteries, arterioles and capillaries or related complaint. Accordingly, the invention involves the administration of stem cells as a treatment or prevention of any one or more of these conditions or other conditions involving weakness in the heart, as well as compositions for such treatment or prevention.

The pharmaceutical compositions disclosed herein are administered via injection, including, but not limited to subcutaneous or parenteral including intravenous, intraarterial, intramuscular, intraperitoneal, intramyocardial, transendocardial, transepicardial, intranasal administration as well as intrathecal, and infusion techniques.

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Moreover, for human administration, preparations should meet sterility, pyrogenicity, general safety and purity standards as required by FDA Office of Biologics standards. The formulations are easily administered in a variety of dosage forms such as injectable solutions, drug release capsules and the like.

In accordance with the present invention, the stem cells are combined with the carrier in any convenient and practical manner, i.e., by solution, suspension, emulsification, admixture, encapsulation, absorption and the like. Such procedures are routine for those skilled in the art.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In a further embodiment of the present invention, the stem cells are combined or mixed thoroughly with a semi-solid or solid carrier. The mixing can be carried out in any convenient manner such as grinding. Stabilizing agents can be also added in the mixing process in order to protect the composition from loss of therapeutic activity. Examples of stabilizers for use in an the composition include buffers, amino acids such as glycine and lysine, carbohydrates such as dextrose, mannose, galactose, fructose, lactose, sucrose, maltose, sorbitol, mannitol, etc., proteolytic enzyme inhibitors, and the like. An example of a semi-solid or solid carrier includes a matrix or gel polymer or gel ointment in which the stem cells are combined resulting in a composition that can be used to graft the cells onto the myocardium of a subject.

III. Treatment of Cardiovascular Disease

Embodiments of the present invention include the methods of administering to a subject isolated cardiac stem cells or a pharmaceutical composition comprising cardiac stem cells to treat cardiovascular disease.

Cardiovascular diseases and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), myocardial infarction and ischemic heart disease. Yet further, one skill in the art recognizes that cardiovascular diseases and/or disorders can result from congenital defects, genetic defects, environmental influences (i.e., dietary influences, lifestyle, stress, etc.), and other defects or influences.

Accordingly, the invention involves the administration of stem cells or a pharmaceutical composition of the present invention as a treatment or prevention of any one or more of these conditions or other conditions involving weakness in the heart, as well as compositions for such treatment or prevention. It is envisioned one of skill in the art will know the most advantageous routes of administration depending upon the disease. In specific embodiments, it is contemplated that the stem cells or pharmaceutical composition can be administered via injection, which includes, but is not limited to subcutaneous, intravenous, intraarterial, intramuscular, intraperitoneal, intramyocardial, transendocardial, transepicardial, intranasal and intrathecal.

Yet further, it is envisioned that the stem cells or pharmaceutical composition of the present invention can be administered to the subject in an injectable formulation containing any compatible carrier, such as various vehicles, adjuvants, additives, and diluents. Adjuvants and/or additives can include, growth factors, cytokines, vitamins, hormones, cardiac transcription factors or any other factor that can enhance cell differentiation and/or increase cell survivability in vivo. Yet further, the stem cells or pharmaceutical composition can be administered parenterally to the subject in the form of slow-release subcutaneous implants or targeted delivery systems such as monoclonal antibodies, iontophoretic, polymer matrices, liposomes, and microspheres.

Treatment regimens may vary as well, and often depend on the cardiovascular disease or disorder, disease progression, and health and age of the subject. Obviously, certain types of cardiovascular disease will require more aggressive treatment, while at the same time, certain patients cannot tolerate more taxing protocols. The clinician will be best suited to make such decisions based on the known efficacy and toxicity (if any) of the therapeutic formulations.

Suitable regimes for initial administration and further doses or for sequential administrations also are variable, and may include an initial administration followed by subsequent administrations; but nonetheless, may be ascertained by the clinician.

For example, the stem cells or the pharmaceutical composition of the present invention can be administered initially, and thereafter maintained by further administration. For instance, a composition of the invention can be administered in one type of composition and thereafter further administered in a different or the same type of composition. For example, a composition of the invention can be administered by intravenous injection to bring blood levels to a suitable level. The subject's levels are then maintained by a subcutaneous implant form, although other forms of administration, dependent upon the subject's condition, can be used.

As used herein the term “effective amount” is defined as an amount of the stem cells or pharmaceutical composition of the present invention that will repair damaged myocardium, regenerate cardiomyocytes, regenerate vascular cells, provide structural stability to an injured myocardium or provide at least partially restored functionality to an injured myocardium. Thus, an effective amount is an amount sufficient to detectably and repeatedly ameliorate, reduce, minimize or limit the extent of the disease or its symptoms.

Cardiac or myocardium structure and function can be measured by various parameters including, but not limited to, left ventricular mass: body weight ratio; changes in cardiomyocyte number, size, mass, and organization; changes in cardiac gene expression; changes in cardiac function; fibroid deposition; changes in dP/dT, i.e., the rate of change of the ventricular pressure with respect to time; calcium ion flux; stroke length; and ventricular output.

The precise determination of what would be considered an effective dose may be based on factors individual to each subject, including their size, age, size of the infarct, and amount of time since damage. Therefore, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Of course, for any composition to be administered to an animal or human, and for any particular method of administration, it is preferred to determine the toxicity, such as by determining the lethal dose (LD) and LD₅₀ in a suitable animal model i.e., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

In further embodiments, the stem cells are administered to a subject suffering from myocardial infarction. It is envisioned that the cells differentiate into at least one cardiac cell type selected from the group consisting of myocytes, endothelial cells, vascular smooth muscle cells, and fibroblasts. It is contemplated that the differentiated cells can alleviate the symptoms associated with myocardial infarction. For example, the injected cells migrate to the infarcted myocardium. The migrated stem cells differentiate into myocytes. The myocytes assemble into myocardium tissue resulting in repair or regeneration of the infarcted myocardium.

Further embodiments of the present invention involve a method of targeting injured myocardium by administering to the cardiac stem cells of the present invention, wherein the cells migrate or home and attach to the injured myocardium. The stem cells are administered intravenously to the subject. Thus, the stem cells maneuver the systemic circulation and migrate or target or home to the damaged or injured myocardium. Once the stem cells have migrated to the damaged myocardium, the stem cells differentiate into myocytes, smooth muscle cells or endothelial cells. It is well known by those of skill in the art that these cell types are essential to restore both structural and functional integrity to a damaged myocardium. Thus, targeting the myocardium with the stem cells of the present invention results in repair of a damaged myocardium.

In further embodiments, the present invention involves a method of repairing injured coronary vessels by administering to the subject an effective amount of cardiac stem cells such that the amount results in regeneration of coronary vascular cells to repair the coronary vasculature.

Another embodiment of the present invention comprises a method of treating heart failure in a subject comprising the step of administering to the subject an effective amount cardiac stem cells that are c-kit^{neg}/CD31⁺/CD38⁺ and express telomerase reverse transcriptase, wherein the amount is effective in at least partially restoring cardiac function. Heart failure can be considered essentially as a progressive disease of apoptotically-mediated cardiomyocyte loss that eventually results in an impaired functional capacity of the cardiac muscle. Thus, it is envisioned that administration of the stem cells of the present invention may at least partially counteract the loss of cardiomyocytes due to apoptosis by replenishing or restoring the cardiomyocytes, thus leading to restoration of cardiac function. The restoration of cardiomyocytes may treat and/or minimize the heart failure suffered by the subject.

A further embodiment is a method of modulating the loss of cardiomyocytes in a subject comprising the step of administering to the subject an effective amount cardiac stem cells that are c-kit^{neg}/CD31⁺/CD38⁺ and express telomerase reverse transcriptase, wherein the amount is effective in at least partially restoring cardiomyocytes. A loss of cardiomyocytes can be related to heart failure and apoptosis. It is envisioned that administration of the cells to a subject that has suffered a loss of cardiomyocytes may treat and/or prevent heart failure in the subject.

Another embodiment is a method of generating myocytes. The myocytes can be generated in vivo or in vitro. In a specific embodiment, the myocytes are generated in vitro. In generating myocytes, cardiac stem cells are obtained from a source, for example, bone marrow, umbilical cord blood, umbilical tissue, left atrial appendage, cardiac tissue, circulating endothelial progenitor cells, cardiac fibroblasts, adipose tissue or skin tissue. The sample can include, the whole tissue, a portion of the tissue or a tissue biopsy from any mammal. Once the stem cells are obtained, they are cultured in vitro so that they differentiate into myocytes. It is envisioned that the stem cells can differentiate into myocytes without the addition of any other factors such as transcription factors.

Yet further, it is also contemplated that factors that are necessary for cardiac development, for example, Nkx2.5 or factors that enhance BMP or Wnt/β-catenin signaling pathway, can be administered to the stem cells. The factors can be administered directly to the cultured stem cells. Yet further, the factors can be administered via an expression vector that expresses the factors. Development of expression vectors are well known and used in the art, for example Maniatis et al., 1982. Once the expression vector is generated it can be delivered to the cells via standard transfection protocols, which are known and used in the art. These standard transfection protocols include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

In further embodiments, it is envisioned that additional cardiac factors (for example a transcription factor, or factors that enhance BMP or Wnt/β-catenin signaling pathway) are administered to a subject to enhance the generation of myocytes in vivo. The transcription factor can be administered via a vector that expresses the transcription factor in vivo. A vector for in vivo expression can be a vector or cells or an expression system, such as a viral vector, i.e., an adenovirus, poxvirus (such as vaccinia, canarypox virus, MVA, NYVAC, ALVAC, and the like), lentivirus or a DNA plasmid vector; and, the cytokine can also be from in vitro expression via such a vector or cells or expression system or others such as a baculovirus expression system, bacterial vectors such as E. coli, and mammalian cells such as CHO cells. See, i.e., U.S. Pat. Nos. 6,265,189, 6,130,066, 6,004,777, 5,990,091, 5,942,235, 5,833,975, which are incorporated herein by reference. The transcription factor compositions may lend themselves to administration by routes outside of those stated to be advantageous or preferred for stem cell preparations; but, transcription factor compositions may also be advantageously administered by routes stated to be advantageous or preferred for stem cell preparations.

Yet further, it is envisioned that the cardiac stem cells are obtained from an autologous source. The autologous source can be tissue that is obtained from a tissue biopsy. The stem cells are proliferated in vitro to generate an abundance of the autologous stem cells. After a suitable number of cells have been proliferated, the autologous stem cells are administered via an intravenous injection to the subject. The stem cells migrate to the damaged myocardium and begin differentiating into myocytes. It is also envisioned that the stem cells or pharmaceutical composition of the present invention can be administered as a prosthesis, such as ex vivo tissue. For example, cells can be isolated from the subject and grown in the form of cylinders or sheets on a matrix (such as scaffold) and surgically introduced into the heart the subject.

It is well known by those of skill in the art that the use of autologous stem cells will reduce and/or eliminate an immune reaction that may occur if allogeneic or xenogeneic stem cells are used. Allogeneic or xenogeneic cells are initially recognized by the subject's immune system through antigenic determinants expressed on the surface of the cells. The predominant antigens recognized as “non-self” are major histocompatibility complex class I and class II antigens (MHC class I and class II). However, if non-autologous stem cells are used, one of skill in the art is aware of the various procedures that may be used to reduce the immune reaction to the stem cells.

One such procedure that is routinely used to inhibit rejection of transplanted cells by the immune system of the subject is the administration of drugs that suppress the function of the immune system. While drugs, such as cyclophosphamide and cyclosporin, effectively inhibit the actions of the immune system, and thus allow acceptance of the cells, their use can cause generalized, non-specific immunosuppression which leaves the subject susceptible to other disorders such as infection. Additionally, administration of immunosuppressive drugs is often accompanied by other serious side effects such as renal failure hypertension.

Still further, hormones or drugs that are known to recruit and activate cells that are resident in the heart under normal conditions can be administered to the subject such that the hormones or drugs recruit and/or activate the resident cells in the heart to divide and generate additional cardiomyocytes.

Another procedure that is readily available to those of skill in the art is to genetically modify the stem cells. Such genetic modification includes, for example altering at least one of the surface antigens to decrease the recognition of non-self, for example see U.S. Pat. No. 5,679,340, which is incorporated herein by reference. Further modifications can also included, packaging of the cells in a liposome, a micelle or other vehicle to mask the cells from the immune system. Thus, one of skill in the art is cognizant of various procedures and techniques that are available to alter a composition so that it is not recognized as “non-self”, thus decreasing the immune response to allogeneic or xenogeneic cell transplantation.

IV Combined Cardiovascular Disease Treatments

In order to increase the effectiveness of the stem cells or pharmacological composition, it may be desirable to combine these compositions and methods of the invention with a known agent effective in the treatment of vascular or cardiovascular disease or disorder. In some embodiments, it is contemplated that a conventional therapy or agent, including but not limited to, a pharmacological therapeutic agent, a surgical therapeutic agent (i.e., a surgical procedure) or a combination thereof, may be combined with the stem cells or the pharmacological composition of the present invention. In a non-limiting example, a therapeutic benefit comprises repair of myocardium or vascular tissue, reduced restenosis following vascular or cardiovascular intervention, such as occurs during a medical or surgical procedure.

This process may involve contacting the cell(s) with an agent(s) and the stem cells or pharmacological composition of the present invention at the same time or within a period of time wherein separate administration of the stem cells and an agent to a cell, tissue or organism produces a desired therapeutic benefit. The terms “contacted” and “exposed,” when applied to a cell, tissue or organism, are used herein to describe the process by which the stem cells and/or therapeutic agent are delivered to a target cell, tissue or organism or are placed in direct juxtaposition with the target cell, tissue or organism. The cell, tissue or organism may be contacted (i.e., by administration) with a single composition or pharmacological formulation that includes both a stem cells and one or more agents, or by contacting the cell with two or more distinct compositions or formulations, wherein one composition includes a the stem cells and the other includes one or more agents.

The treatment may precede, be co-current with and/or follow the other agent(s) by intervals ranging from minutes to weeks. In embodiments where the stem cells, and other agent(s) are applied separately to a cell, tissue or organism, one would generally ensure that a significant period of time did not expire between the time of each delivery, such that the stem cells and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the stem cells. In other aspects, one or more agents may be administered within from substantially simultaneously, about minutes to hours to days to weeks and any range derivable therein, prior to and/or after administering the stem cells.

Administration of the stem cell composition to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an

B. Surgical Therapeutic Agents

In certain aspects, a therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Further treatment of the area of surgery may be accomplished by perfusion, direct injection, systemic injection or local application of the area with at least one additional therapeutic agent (i.e., the stem cells of the present invention, a pharmacological therapeutic agent), as would be known to one of skill in the art or described herein.

V. Kits

Any of the compositions described herein may be comprised in a kit. In a non-limiting example, the stem cells, lipid, and/or additional agent, may be comprised in a kit. The kits will thus comprise, in suitable container means, the stem cells and a lipid, and/or an additional agent of the present invention.

The kits may comprise a suitably aliquoted stem cells, lipid and/or additional agent compositions of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the stem cells or the pharmacological composition of the present invention, lipid, additional agent, and any other reagent containers in close confinement for commercial sale. Such containers may include injection or blow-molded plastic containers into which the desired vials are retained.

Therapeutic kits of the present invention are kits comprising the stem cells. Such kits will generally contain, in suitable container means, a pharmaceutically acceptable formulation of the stem cells. The kit may have a single container means, and/or it may have distinct container means for each compound.

When the components of the kit are provided in one and/or more liquid solutions, the liquid solution is an aqueous solution, with a sterile aqueous solution being particularly preferred. The stem cell compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the stem cells are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, i.e., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate the stem cell composition within the body of an animal. Such an instrument may be a syringe, pipette, forceps, and/or any such medically approved delivery vehicle.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Flow Cytometry and Magnetic Enrichment

A total cardiac cell preparation was isolated from 6-12 week-old C57BL/6 mice by intracoronary perfusion with 0.025% collagenase, as necessary for viable adult mouse cardiomyocytes (Zhou et al., 2000).

Yet further, a myocyte-depleted population was prepared, incubating minced myocardium in 0.1% collagenase (30 min. 37° C.), which is lethal to most adult mouse cardiomyocytes (Zhou et al., 2000). Dissociated cells were filtered through 70 μm mesh. Bone marrow cells (Goodell et al., 1996) were compared, with or without 0.1% collagenase and filtration. Cells were immunolabeled using Sca-1-phycoerythrin (PE), Sca-1-fluorescein isothiocyanate (FITC), c-kit-PE, CD4-FITC, CD8-FITC, B220-FITC, Gr-1-FITC, Mac-1-FITC, TER-119-FITC, CD45-FITC, CD31-FITC, CD38-FITC, Flk-1-FITC, VE-cadherin-biotin, vWf-biotin and Flt-1. CD45-PE11 was used for bone marrow cells. Biotinylated antibodies were detected with streptavidin-PE or streptavidin-FITC, Flt-1 with FITC-conjugated secondary antibody, and non-viable cells with propidium iodide. Flow cytometry was performed with an EPICSXL-MCL (Beckman Coulter). Gates were established by the non-specific Ig binding in each experiment.

For purification, dissociated cells labeled with Sca-1-biotin were incubated with anti-biotin microbeads and purified by 5-6 cycles with positive and negative selection columns (McKinney-Freeman et al., 2002). The first depleted fraction was used for Sca-1− cells. Each cycle of positive selection conferred ˜5% enrichment for Sca-1+ cells, above the 75% at first pass. Magnetically sorted populations for gene expression and cell grafting were routinely reanalyzed by flow cytometry and the purity of Sca-1+ cells confirmed.

Efflux of Hoechst dye 33342 was used to define the side population (SP) cells that have been reported to be enriched for repopulating cells in bone marrow (Goodell et al., 1997) and other tissue (Welm et al., 2002; Asakura et al., 2002).

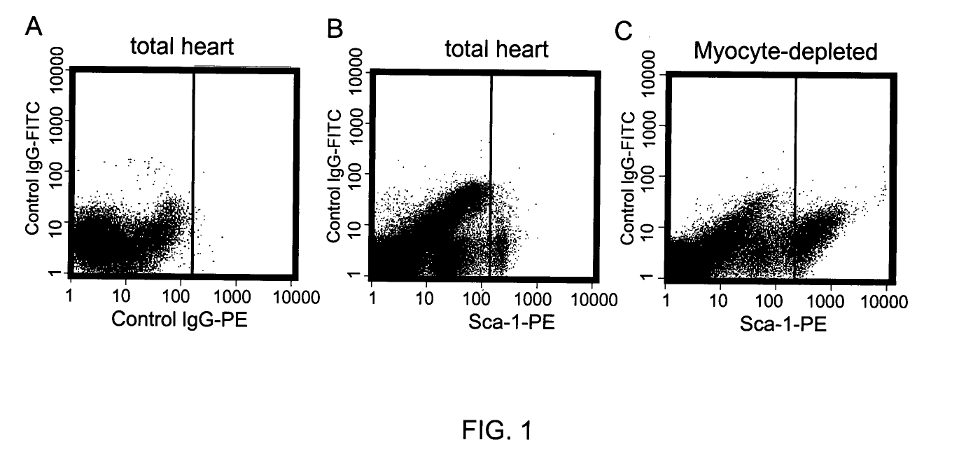

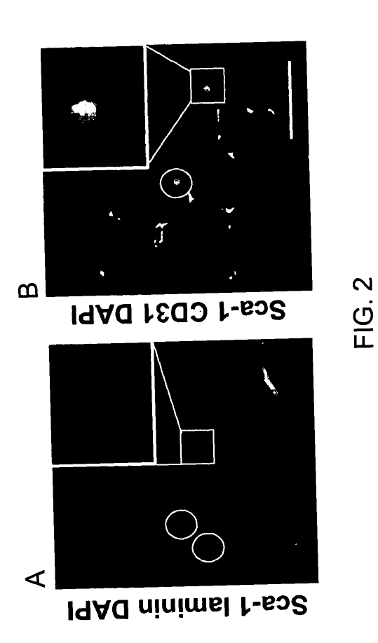

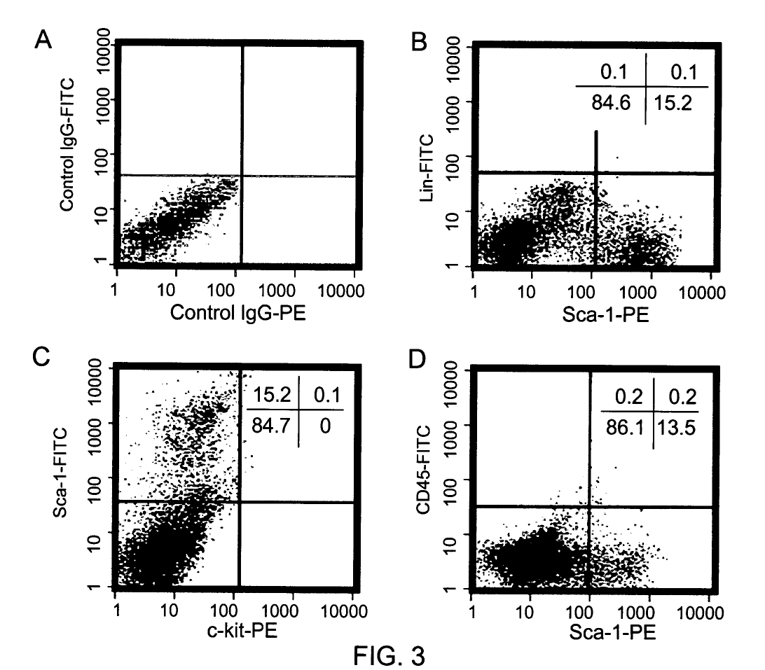

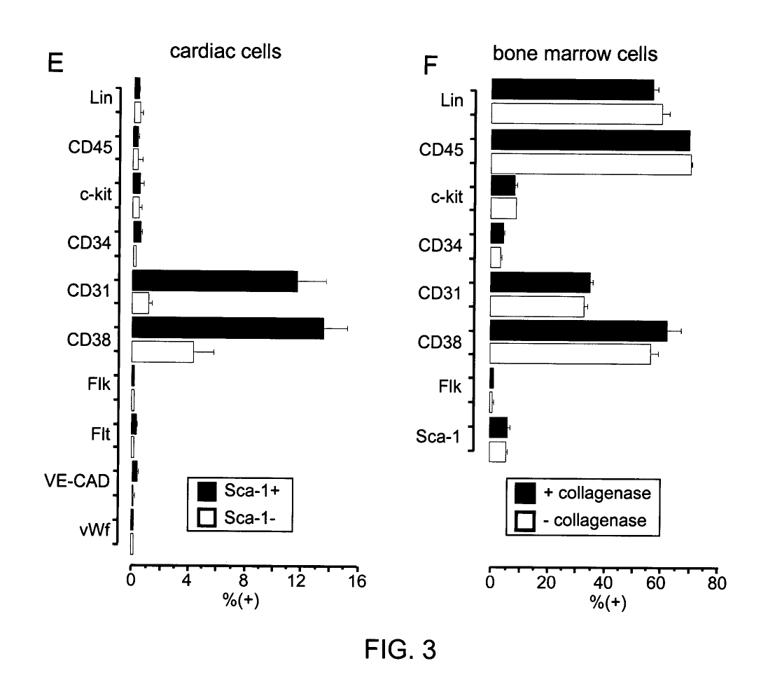

Approximately 14-17% of the cells expressed Sca-1 (enriched 7-fold, compared to total cardiac cells; FIG. 1A-FIG. 1C). As found for Sca-1+ cells in skeletal muscle (Asakura et al., 2002), cardiac Sca-1+ cells were small interstitial cells, often in proximity with cells expressing CD31/platelet endothelial cell adhesion molecule-1 or its receptor CD38, which is implicated in cell binding (FIG. 2A-FIG. 2B). Cardiac Sca-1+ cells lacked blood cell lineage markers (CD4, CD8, B220, Gr-1, Mac-1, TER119), c-kit, Flt-1, Flk-1, VE-cadherin, von Willebrand factor (vWf), and the hematopoietic stem cell markers CD45 and CD34 (FIG. 3A-FIG. 3E). Levels of the markers in bone marrow cells, assayed as a control, were not diminished by collagenase (FIG. 3F), excluding false-negative results after enzymatic digestion. These features argue against a hematopoietic progenitor cell, endothelial progenitor cell, or mature endothelial phenotype.

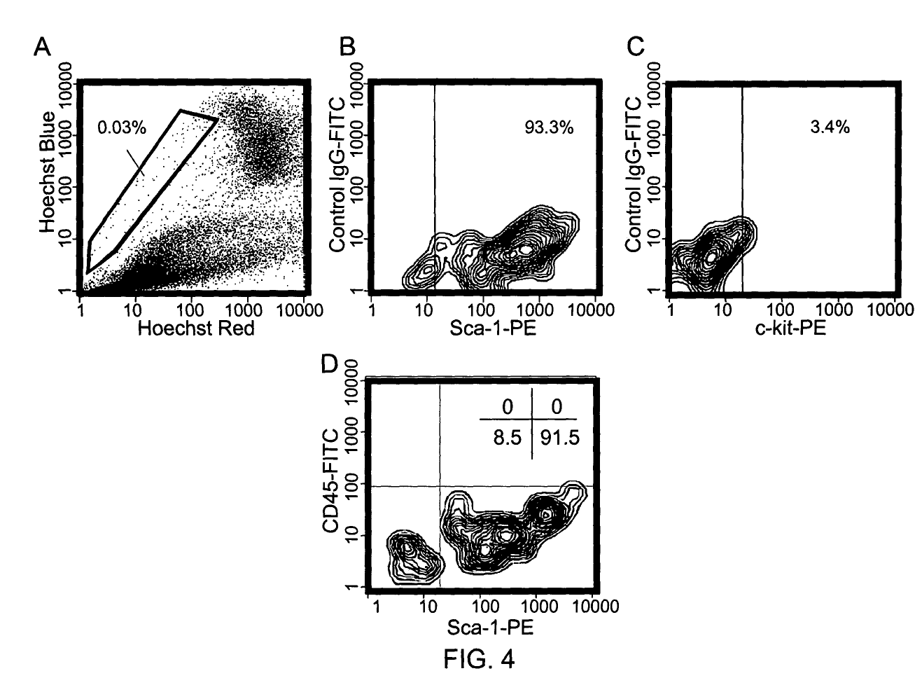

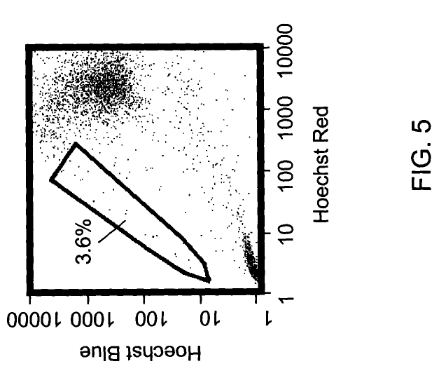

There were heterogeneities within this uncloned population, for example, most but not all Sca-1+ cells expressed CD31 or its receptor CD38, which was implicated in cell-cell binding as shown in FIG. 2 and FIG. 3. A smaller fraction (0.03%) of cardiac cells possessed the dye-exclusion properties of SP cells (FIG. 4A-FIG. 4D). The prevalence of SP cells even in bone marrow was only 0.05%, yet accounted for much or all of the long-term self-renewing cells (Goodell et al., 1996). SP cells from myocardium were >93% Sca-1+, which differed from marrow SP cells by typically lacking CD45 and c-kit (FIG. 4A-FIG. 4D) (Jackson et al., 2001), and were enriched 100-fold in the Sca-1+ population (FIG. 5).

### Example 2

### Telomerase Expression

Telomerase reverse transcriptase (TERT) is associated with self-renewal potential, down-regulated in adult mouse myocardium, and sufficient to prolong cardiomyocytes cycling (Oh, et al., 2001). Thus, TERT expression was measured in the isolated Sca-1+ and Sca1-1 fractions.

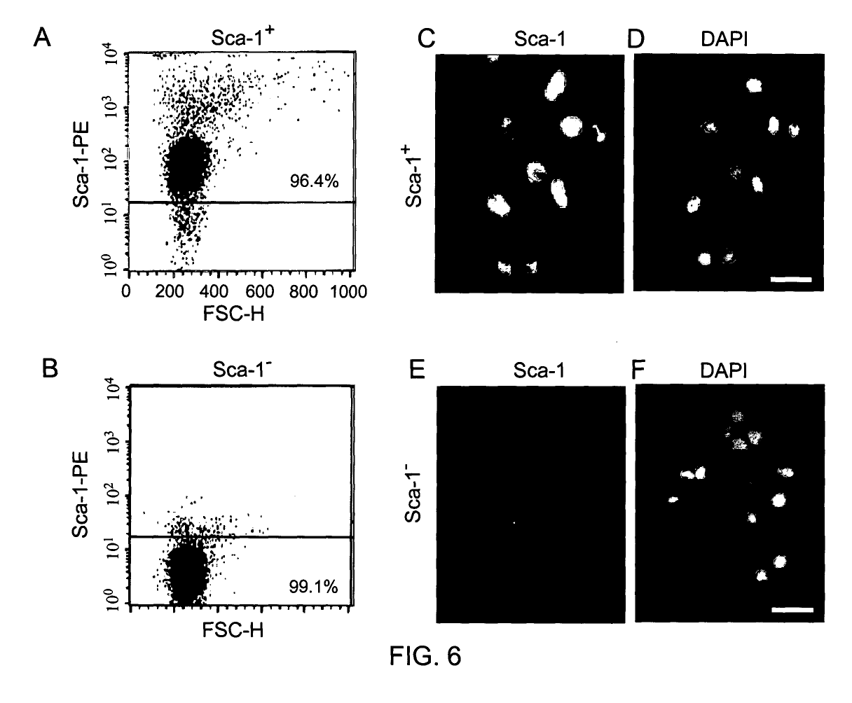

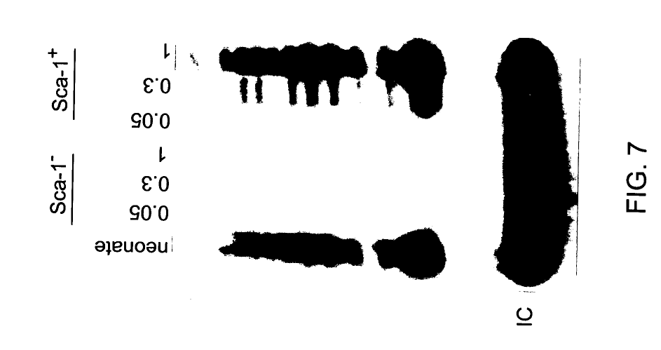

Briefly, a Sca-1+ fraction (>96% pure, after 5 or more rounds) and Sca-1− fraction (>99% pure, even in the flow-through; FIGS. 6A-6F) was isolated as described in Example 1. By a telomeric repeat amplification protocol (Oh et al., 2001), telomerase activity was detected only in Sca-1+ cells from adult heart, not Sca-1− cells, at levels similar to neonatal myocardium (FIG. 7). Analogously, >60% of Sca-1+ cells co-stained for TERT, but not Sca-1-cells.

### Example 3

### Gene Expression

RNA was isolated was isolated and assayed by RT-PCR (Soonpaa et al., 1996). The primers used include: CRP1, (SEQ.ID.NO.1) GGAAGAGGTGCAGTGCGATG forward, (SEQ.ID.NO.2) ACCTGGAACACTTCTCAGCT reverse; CRP2, (SEQ.ID.NO.3) GGAAGAGGTGCAGTGCGATG forward, (SEQ.ID.NO.4) ACCTGGAACACTTCTCAGCT reverse; CRP3, (SEQ.ID.NO.5) GGAAGAGGTGCAGTGCGATG forward, (SEQ.ID.NO.6) ACCTGGAACACTTCTCAGCT reverse; SRF, (SEQ.ID.NO.7) CCTTTTCACGGTT TCTTTACACACACACTG forward, (SEQ.ID.NO.8) GGTCAGCTAATACTCATAGCA AATTCAGCC reverse. The remaining primers were known in the art (Jackson et al., 2001; Makino et al., 1999; Gaussin et al., 2002; Yamashita et al., 2000).

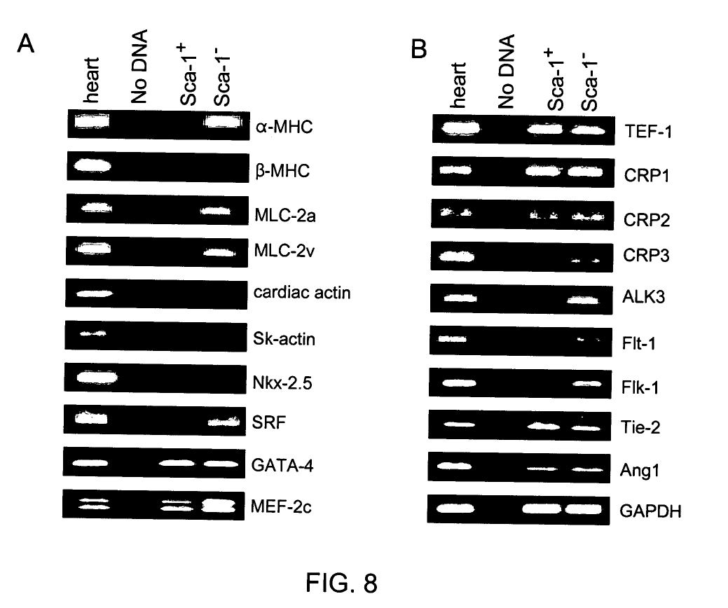

Cardiac genes were measured using quantitative RT-PCR and corrected for glyceraldehyde-3-phosphate dehydrogenase (GAPDH). As shown in FIG. 8A and FIG. 8B, Sca-1+ cells expressed none of the following cardiac genes: α and β-myosin heavy chain (MHC), atrial and ventricular myosin light chain-2 (MLC-2a, -2v), cardiac and skeletal α-actin, and muscle LIM protein/cysteine-rich protein-3 (MLP/CRP3). Most were readily detected in Sca-1− cells, consistent with the presence of differentiated myocytes in the starting myocyte-depleted fraction. Consistent with the lack of all late myocyte markers, Sca-1+ cells did not express the cardiac Hox gene Nkx2.5, and had minimal levels of SRF. However, GATA-4, MEF-2C, and TEF-1, encoding other cardiogenic transcription factors, were expressed. Notably, these also were expressed in undifferentiated marrow stromal cells (Makino et al., 1999). Consistent with the absence of Flt-1 and Flk-1 protein by flow cytometry (FIG. 3E), little or no expression was seen in Sca-1+ cells by RT-PCR (FIGS. 8A-8B). Sca-1+ and Sca-1− cells did express Tie-2 and angiopoietin-1 (Ang1) mRNA, which is found not just in the developing vasculature, but also in some SP cells (Jackson et al., 1999).

### Example 4

### Gene Expression by Microarray Analysis

Expression profiling was preformed using Affymetrix MG U74Av2 microarrays, an Agilent GeneArray Scanner, Affymetrix Microarray Suite version 5.0, and dChip 1.2.

Microarray expression profiling was concordant with the results obtained from PCR in Example 3, extending the cardiac structural genes that were not expressed in cardiac Sca-1⁺ cells, and adding Bop and popeye-3 to Nkx2.5 as cardiogenic transcription factors that are absent (Table 1). Beyond the lack of CD45, CD34, c-kit, and Flt-1 predicted by flow cytometry, it also established the absence of hematopoietic stem cell transcription factors in Sca1-1+ cells (Lmo2, GATA2Tal1/Sc1). Conversely, transcripts detected in adult cardiac Sca-1+ cells but not adult cardiomyocytes were enriched, as expected, for cell cycle regulators, as well as diverse growth factors, cytokines, chemokines, and their receptors (Table 1). Genes enriched in Sca-1+ cells also included multiple transcriptional repressors (DNA methyltransferase-1, histone deacetylase-1, the Notch effector Hesl, Groucho-binding proteins runx1 and runx2), a property of both adult and embryonic stem cells (Ramalho-Santos et al., 2002). Absence of Oct-4 and UTF-1, by mircroarray profiling, was confirmed by RT-PCR.

TABLE 1
Expression profiling of adult cardiac Sca-1+ cells versus cardiomyocytes
Transcripts detected in purified adult cardiac myocytes but not cardiac Sca-1+ cells

Sarcomeric proteins: Acta1, Actc1, Mybpc3, Myhca, Myhcb, Mylc, Mylc2a, Mylpc, Myom1,
Myom2, Tncc, Tnni3, Tnnt1
Transcription factors: Bop, Csrp3, Nkx2-5, Pop 3
Growth factors: Fgf1
Metabolism: Acas2, Adss1, Art1, Ckmt2, Ckmm, Cox6a2, Cox7a1, Cox8b, Crat, Cyp4b1,
Fabp3, Facl2, Mb, Pgam2, Pygm, Slc2a4
Ion transport: Atp1a2, Cacna1s, Casq2, Kcnq1, Kcnj8, Ryr2
Other: Cdh13, Ldb3, Nppb, Sgca, Sgcg
275 transcripts were detected in adult cardiac myocytes but not adult cardiac Sca-1+ cells, of
which relevant transcripts with a eight-fold or more
change in signal intensity are shown. Others include 35 ESTs, 37 RIKEN cDNAs, and 11
unannotated mRNAs.

Transcripts detected in cardiac Sca-1+ cells but not purified adult cardiac myocytes

Growth factors, cytokines, receptors: Adm, Bmp1, Csf1, Crlf1, Fgfr1, Figf, Frzb, Fzd2, Inhba,
Inhbb, Igf1, Igfbp2, Igfbp4, Il4ra, Il6, Pdgfra, Sfrp1,
Scya2, Scya7, Scya9, Scyb5, Sdf1, Tgfb2, Tnfrsf6, Vegfc, Wisp1, Wisp2
Transcription factors: Aebp1, Csrp, Csrp2, Dnmt1, Edr2, Foxc2, Hey1, Hdac1, Madh7, Ndn,
Nmyc1, Odz3, Pias3, runx1, runx2, Tcf21, Twist,
ZBP-99
Cell cycle: Cdc2a, Cks1, Ccnb1-rs1, Ccnc, Ccne2, Prim2, Mki67, MCM7, Rab6kifl, Rev3l,
Rrm1, Tyms, Top2a
Adhesion, recognition: Anxa1, Npnt, Nid2, Ptx3, Tm4sf6, Vcam1
Signal transduction: Borg4, Cask, Ect2, Eif1a, Lasp1, Map3k6, Map3k8, Pscd3, Sphk1, Stk6,
Stk18, Tc10l, Wrch1
Extracellular matrix: Adam9, Mmp3, Col1a1, Col1a2, Col3a1, Col4a5, Col5a, Col5a2, Col8a1,
Lox, Spp1, Timp, Tnc
816 transcripts were detected in adult cardiac Sca-1+ cells but not adult heart, of which, relevant
transcripts with a eight-fold or more change in signal
intensity are shown. Others include 116 ESTs, 154 RIKEN cDNAs, and 28 unannotated mRNAs.

### Example 5

### In Vitro Differentiation of Cardiac Cells

Freshly isolated cardiac Sca-1+ cells were grown in 35-mm dishes coated for 1 hr with 200 μg/mL fibronectin (Sigma) and maintained in Medium-199, 10% FBS, for 3 days (5% CO₂, 37° C.). To induce differentiation, cells were cultured in medium containing 2% FBS and 3 μM 5-aza (3 days) (Makino et al., 1999) or 1% DMSO (1 week) (Monzen et al., 1999). Cells were photographed using a Zeiss Axioplan 2.

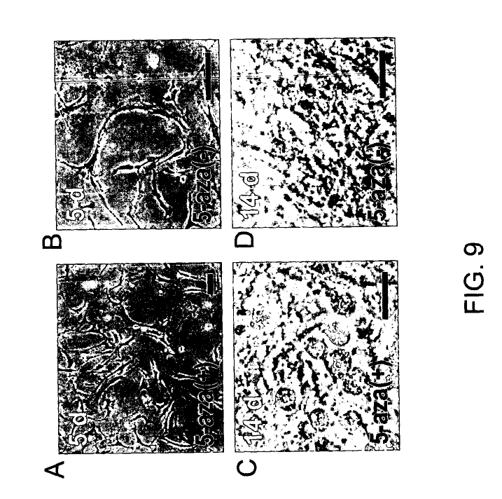

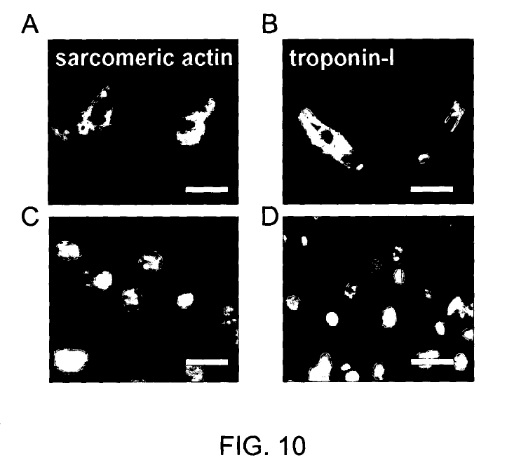

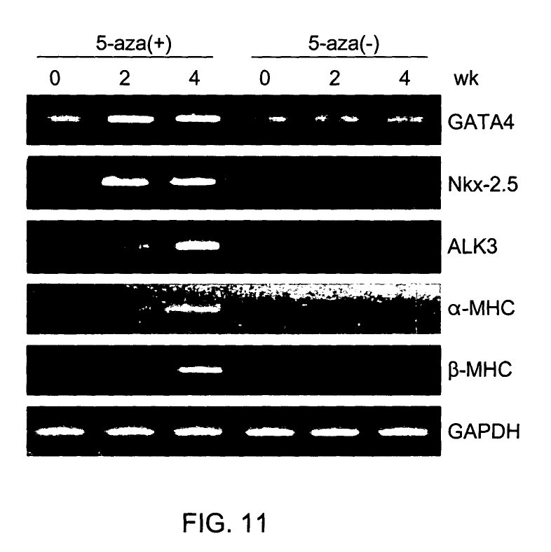

In the presence of 5-aza, Sca-1+ cells gradually developed multicellular spherical structures that were tightly adherent to the monolayer (FIGS. 9A-9D) then flattened after 2 wk in culture. Immunostaining at 4 wk confirmed the induction of sarcomeric α-actin (4.6±1.2%) and cardiac troponin-I (2.8+0.9%) in treated cells (FIGS. 10A-10D) but not untreated ones. Nkx-2.5, αMHC, βMHC, and Bmpr1a, a receptor for bone morphogenetic proteins involved in heart development (Schneider et al., 2003), were each highly induced by 5-aza (FIG. 11), all but βMHC was apparent at 2 wk. None of the genes were expressed in the absence of 5-aza even after 4 wk, or in cells treated with 1% dimethylsulfoxide.

### Example 6

### Cardiac Cell Culture and Viral Gene Transfer

Sca-1+ cells from Bmpr1a^{F/-} (containing one loxP-flanked and one null allele) hearts were isolated as described in Example 1. The isolated cells were grown in 35-mm tissue culture dishes coated with 200 μg/mL fibronectin, and were maintained in Medium-199 containing 10% FBS for 3 days in 5% CO₂ at 37° C. To induce differentiation, cells were cultured in 2% FBS plus 3 μM 5-aza (3 days) or 1% DMSO (1 week). To disrupt the conditional Bmpr1a allele, adenovirus encoding LacZ versus Cre (20 PFU/ml) was added on day 3 for 6 hr in serum free medium (Agah et al., 1997), before giving 5-aza or DMSO. DNA was extracted 24 hr after infection, and recombination tested using primers external to the paired loxP motifs (Gaussin et al., 2002).

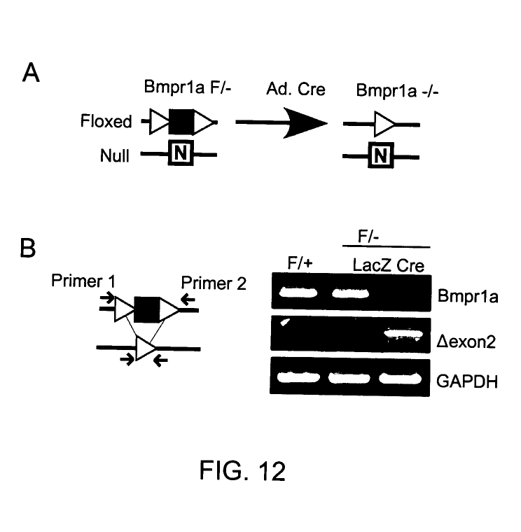

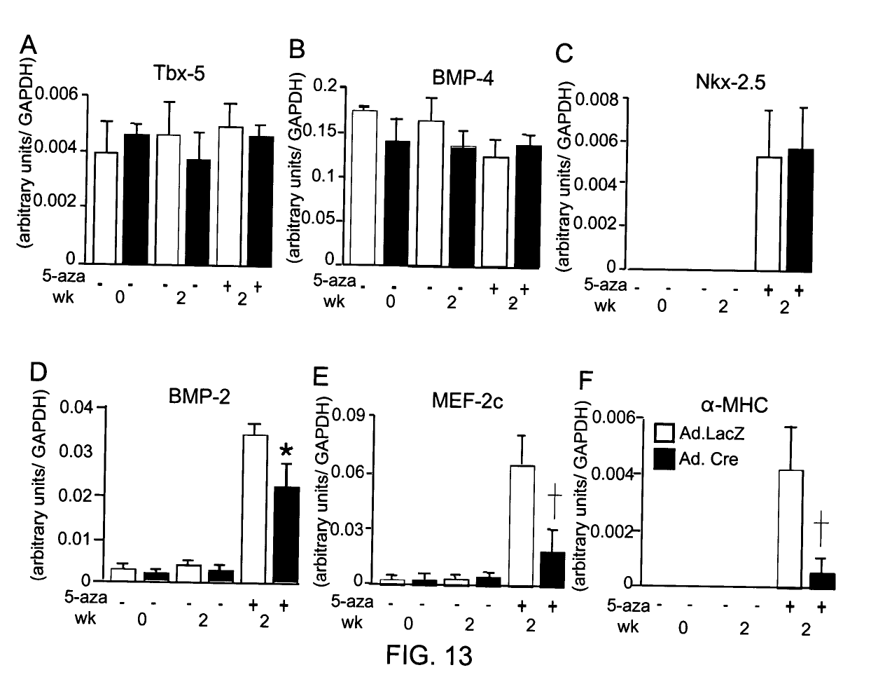

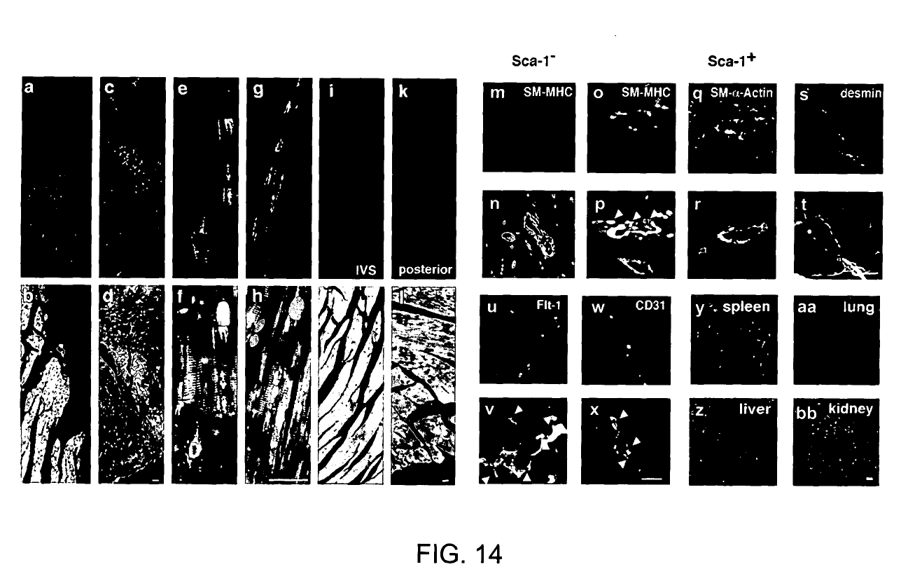

Disruption of Bmpr1a by Cre was confirmed by PCR (FIG. 12). Differentiation by 5-aza, with and without Bmpr1a, was compared using quantitative RT-PCR (FIG. 13A-FIG. 13F). Neither Tbx5 (FIG. 13D) nor BMP-4 (FIG. 13B) required 5-aza for expression, and their expression was unchanged by the loss of ALK3. By contrast, deletion of Bmpr1a significantly impaired the induction of BMP-2 (FIG. 13A), MEF-2C (FIG. 13E), and, especially, αMHC (FIG. 13F). Morphologically, these cells resembled Sca-1+ cells without 5-aza. Among the genes investigated, only Nkx-2.5 was induced by 5-aza, yet unaffected by disruption of Bmpr1a.

### Example 7

### Immunostaining and Western Blot

To localize Sca-1 plus laminin, monoclonal Sca-1 antibody was conjugated with Alexa Fluor 495; sections were counterstained with rabbit anti-laminin, then FITC-conjugated goat anti-rabbit IgG (Sigma). To localize Sca-1 plus CD31, monoclonal antibody to PECAM was conjugated with Alexa Fluor 495; sections were counterstained with Sca-1-FITC. Potential mosaicism of R26R was assessed by immunostaining with rabbit antibody to neo. To detect LacZ activation by Cre+ donor cells, myocytes were stained using mouse monoclonal antibody to β-galactosidase, then Texas Red conjugated goat antibody to mouse IgG; cells were counter-stained with FITC-conjugated mouse antibody to sarcomeric α-actin or rabbit antibody to laminin, then FITC-conjugated goat antibody to rabbit IgG. To elucidate the prevalence of fusion more precisely, myocardium was triply stained for Cre, LacZ, and neo, using mouse antibody to Cre (BabCO) conjugated with Alexa Fluor 488, mouse antibody to β-galactosidase conjugated with Alexa Fluor 594, rabbit antibody to neo, and goat anti-rabbit IgG conjugated with Alexa Fluor 647. Nuclei were counterstained with 4′, 6-diamidino-2-phenylindole (DAPI). Mouse antibody to sarcomeric actin was conjugated with Alexa Fluor 647 and mouse antibody to connexin-43 with Alexa Fluor 495. Mitotic phosphorylation of histone H3 was detected using rabbit antibody to the serine-10 phospho-epitope, then goat anti-rabbit IgG conjugated with Alexa Fluor 647. Irrelevant mouse and rabbit antibodies conjugated with each fluor were used as the negative controls. Immunostaining was visualized by confocal microscopy. Western blotting for neo was performed using a 1:1000 dilution of neo antibody, versus total actin as a control.

### Example 8

### Myocardial Infarction and Cell Delivery

Cell grafting was performed with a Cre/lox (αMHC-Cre/R26R) donor/recipient pair (Agah et al., 1997; Soriano et al., 1999). Ischemia/reperfusion injury was performed in chronically instrumented, closed chest R26R mice, using an implantable occluder 45. Four to 5 days after instrumentation, the left anterior descending coronary artery was occluded for 1 hr, with reperfusion for 6 hr. Freshly isolated Sca-1+ cells (10⁶) from αMHC-Cre mice or wildtype littermates were injected in 100 μl PBS via the right jugular vein. Mice were euthanized 2 weeks later, with comparable survival (62%) in each group.

### Example 9

### Homing and Differentiation of Cardiac Cells

Purified cardiac Sca-1+ cells were labeled for 15 min with the lipophilic green fluorescent membrane dye, PKH2-GL (4×10⁻⁶ M) and washed 4× to remove unincorporated dye. The labeling efficiency was >98%, and persisted in culture for more than 2 weeks.

To minimize factitious cytokine responses to acute surgical manipulation, coronary artery ligation and reperfusion were performed in chronically instrumented, closed-chest mice (Nossuli et al., 2000). Four to 5 days after instrumentation, the left anterior descending coronary artery was occluded for 1 hr, followed by reperfusion for 6 hr. PKH2-labeled Sca-1+ cells (0.1-1×10⁶) were then injected in 100 μl PBS via the right jugular vein. Labeled cells also were injected 7 hr after a surgical control (instrumentation, without occlusion). Mice were euthanized 24 hr or 2 weeks after injection, with comparable survival (62%) in each group.

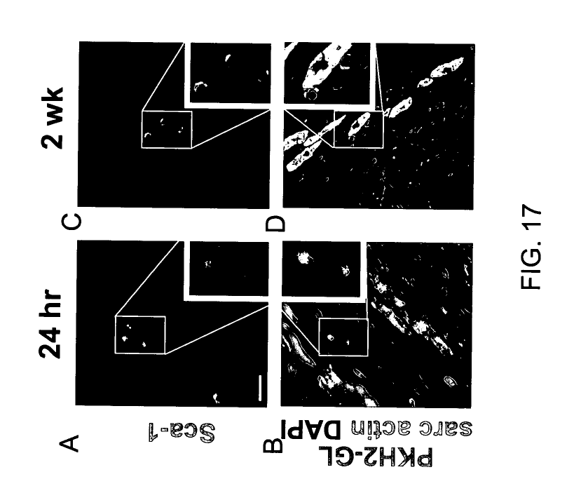

The presence of donor cells in myocardium was detected by epifluorescence microscopy within 24 hr (mean, 0.8±0.5% of total left ventricular cells), and stable engraftment confirmed in 10 of 14 mice at 2 weeks (5.1±1.1, suggesting proliferation in the interium) (FIG. 14 and FIG. 17). Differentiation of donor cells was confirmed by the presence of sarcomeric α-actin (FIG. 14). Differentiated donor myocytes were abundant in the infarct border zone (18.1±4.4% of nuclei), but absent from the infarct itself (FIG. 14A-FIG. 14D). Donor-derived myocytes were contiguous with host myocytes, with no apparent distortion of tissue architecture (FIG. 14E, FIG. 14F). Similar results were obtained for cardiac troponin-I (FIG. 14G, FIG. 14H). No engraftment was seen in the uninfarcted interventricular septum (FIG. 14I and FIG. 14J), posterior wall (FIG. 14K and FIG. 14L), or right ventricle. No systematic variation occurred in levels of engraftment, using 10⁵ to 10⁶ cells. Injection of Sca-1+ cells generated more than 5% of total left ventricular myocytes following infarction, unlike Sca-1− cells (0.1%, p<0.001) but did not produce engraftment without infarction (0.0001%, p<0.0001). Similar numbers resulted for donor-derived endothelial cells (Flt-1+), with 10-fold fewer donor-derived smooth muscle cells, identified by smooth muscle-MHC (SM-MHC; FIG. 14M-FIG. 14X). Cardiac Sca-1+ cells were not detected in lung, liver, or kidney but label was seen in the spleen (FIG. 14Y-FIG. 14bb), as with marrow-derived mesenchymal stem cells.

### Example 10

### Homing, Differentiation and Fusion of Cardiac Cells

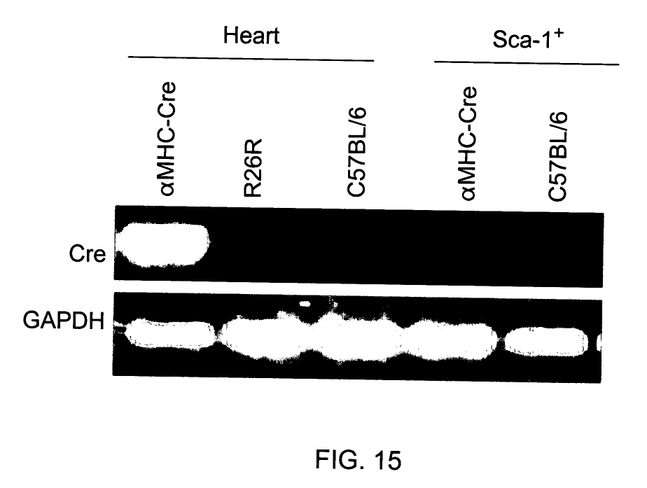

Cardiac Sca-1+ cells from mice expressing Cre recombinase via the αMHC promoter (Agah et al., 1997) were injected into R26R recipients (Soriano et al., 1999). Freshly isolated cardiac Sca1+ cells from αMHC-Cre mice do not express Cre (FIG. 15), as foreseen given their lack of endogenous αMHC (FIGS. 8A and 8B). The R26R reporter line, bearing a Cre-dependent LacZ gene behind a loxP-flanked stop signal, was transcribed ubiquitously without mosaicism (Soriano et al., 1999), and αMHC-Cre mediated efficient recombination at this locus even at the incipient levels in early myocardium (Gaussin et al., 2002).

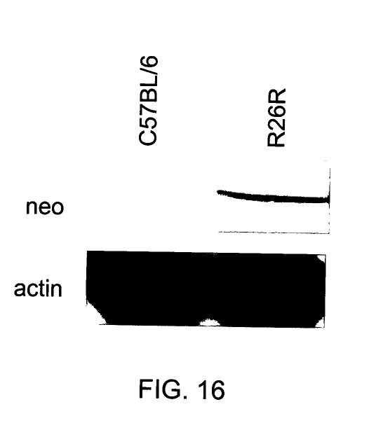

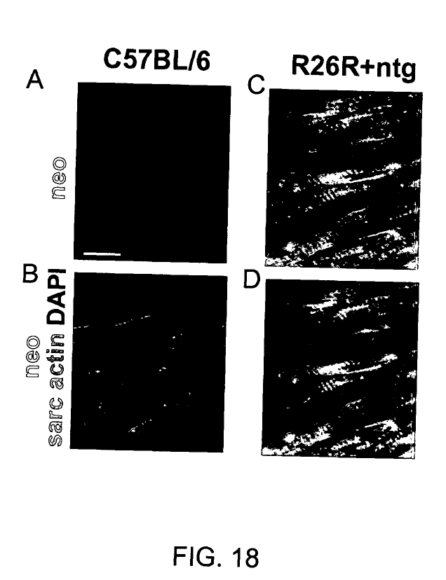

To ascertain the uniformity of R26R expression in adult heart, homogeneity of neomycin phosphotransferase (neo) was tested, which provided the LoxP-flanked “stop” signal upstream from LacZ. Neo was measured in R26R mice by Western blotting (FIG. 16). FIG. 18 also shows that wild-type C57BL/6 mice do not express neo.

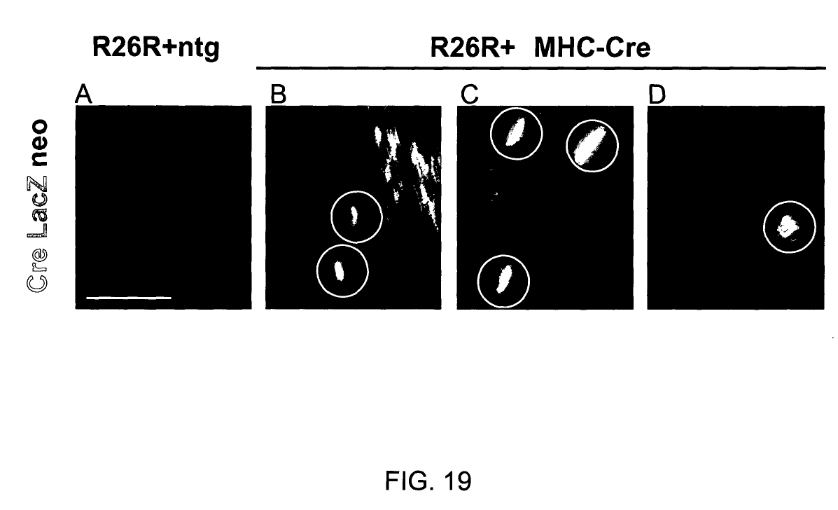

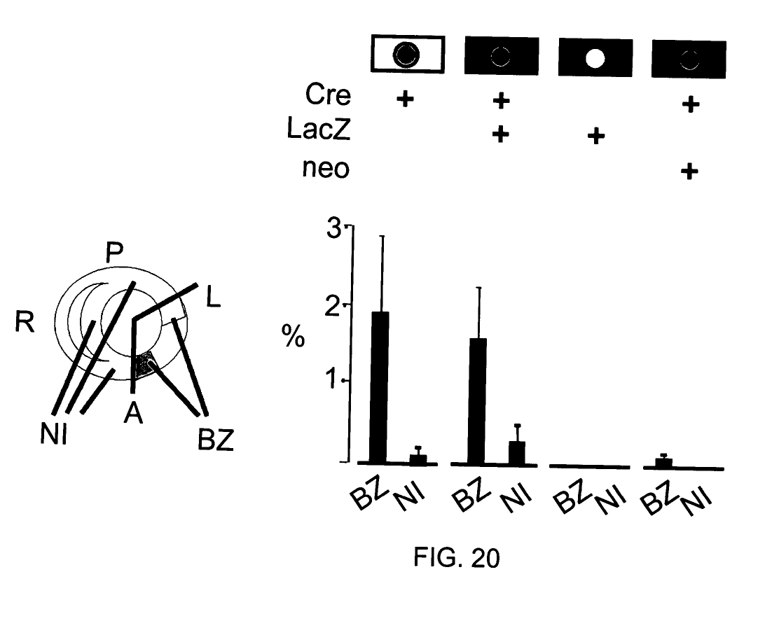

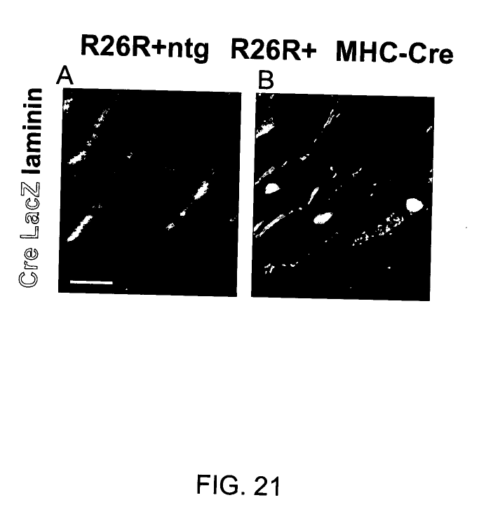

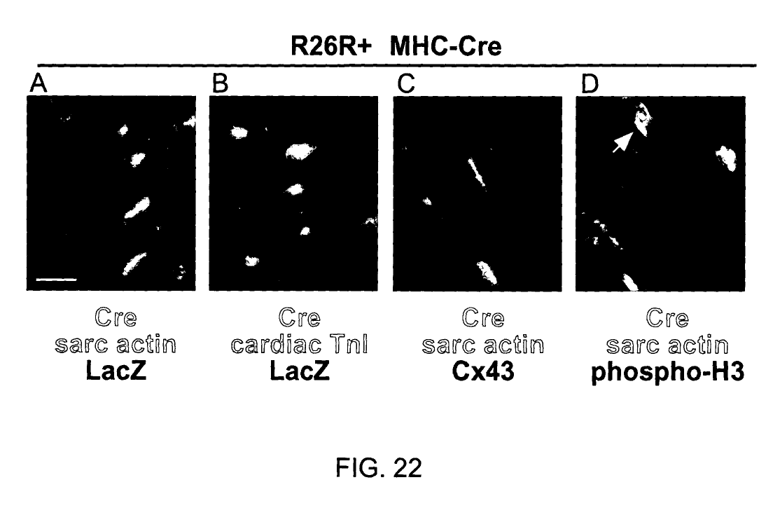

Two weeks after injury and intravenous infusion of undifferentiated αMHC-CreSca-1+ cells, nuclear-localized Cre protein was detected specifically in the infarct border zone (FIG. 19, FIG. 21, and FIG. 22). The prevalence of differentiated, Cre-expressing, donor-derived cells was 3.4±0.8% of total left ventricular myocytes, 150-fold greater than seen for engraftment by endogenous marrow-derived SP cells (Jackson et al., 1999), and localized almost exclusively in anterolateral myocardium, the region subjected to infarction. Co-expression of Cre and LacZ was readily detected as evidence of chimerism (FIG. 20). (Injection of non-transgenic cardiac Sca-1+ cells lacking αMHC-Cre did not produce Cre protein or activate LacZ; FIGS. 19A-19D)). By immunostaining as described in Example 7 for LacZ plus sarcomeric αactin or laminin, LacZ activation was confined to myocytes (FIG. 19, and FIG. 21, respectively).

Conversely, roughly half the cells expressing αMHC-Cre did not co-express LacZ (FIGS. 19-22). Such Cre+LacZ− cells may indicate differentiation autonomous of fusion (bona fide cardiopoiesis) or, alternatively, might be false-negative examples—fused cells with incomplete penetrance for recombination of R26R. By triple staining for Cre plus neo plus LacZ, fused cells without recombination were detected sporadically, but were minuscule in prevalence and did not contribute significantly to the Cre+ population (FIG. 19D).

Hence, fusion-associated and fusion-independent activation of αMHC-Cre both appeared to be operative. As independent criteria of their differentiation in vivo, all donor derived, differentiated (Cre+) cells also expressed sarcomeric α-actin (FIG. 22) and connexin-43 (FIG. 22). Assayed 2 weeks after cell grafting, 5% of Cre+ sarcomeric actin+ cells (41/816) stained for the serine-10 phosphorylation of histone H3, a marker of mitotic Cdc2 activity (Oh et al., 2001), versus only 0.00004% of Cre− cardiomyocytes (1/24,000).

### Example 11

### Generation and Administration of Myocytes from Human Sources In Vitro

Cardiac stem cells are obtained from a human source, for example, bone marrow, umbilical cord blood, umbilical tissue, left atrial appendage, cardiac tissue, circulating endothelial progenitor cells, cardiac fibroblasts, adipose tissue or skin tissue. The sample is a tissue biopsy from an autologous source or homologous source.

Once the stem cells are obtained, they are cultured in vitro so that they differentiate into myocytes. Additional factors can be added to the stem cells to enhance differentiation into myocytes. Factors that are necessary for cardiac development can include Nkx2.5 or factors that enhance BMP or Wnt/β-catenin signaling pathway. The factors can be administered directly to the cultured stem cells. The factors can also be administered via an expression vector that expresses the factors. Once the expression vector is generated it can be delivered to the cells via standard transfection protocols, which are known and used in the art.

The stem cells are administered intravenously or as a prosthesis, such as ex vivo tissue to the subject. Ex vivo tissue refers to cells that are isolated from the subject and grown in the form of cylinders or sheets on a matrix (such as scaffold) and surgically introduced into the heart the subject. The stem cells repair myocardium.

### Example 12

### Generation and Administration of Myocytes from Human Sources In Vivo

Cardiac stem cells are obtained from a human source, for example, bone marrow, umbilical cord blood, umbilical tissue, left atrial appendage, cardiac tissue, circulating endothelial progenitor cells, cardiac fibroblasts, adipose tissue or skin tissue. The sample is a tissue biopsy from an autologous source or homologous source.

Once the stem cells are obtained, they are cultured in vitro to proliferate the cells. After a suitable number of cells have been proliferated, the stem cells are administered to the subject. The stem cells are administered to a subject and additional cardiac factors (for example a transcription factor, or factors that enhance BMP or Wnt/β-catenin signaling pathway) are administered to a subject to enhance the generation of myocytes in vivo.

The stem cells migrate to the damaged myocardium and differentiate into myocytes to repair the myocardium.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.

Agah, R. et al., J. Clin. Invest. 100, 169-179 (1997).

Bishopric N H, et al., Curr Opin Pharmacol. 1(2):141-50, (2001).

Chen and Okayama Mol Cell Biol. 7(8):2745-52 (1987).

Chiu et al., Ann Thorac Surg. 60:12-8 (1995).

Clarke, D. L. et al., Science 288, 1660-1663. (2000).

Cohen et al., J. Immunol. 32:38-42 (1984);

Condorelli, G. et al., Proc Natl Acad Sci USA 98, 10733-10738. (2001).

Cripps, R. M. & Olson, E. N. Dev Biol 246, 14-28. (2002).

Crow, M., Circ Res. 9;91(3):183-5 (2002).

Erolsspm et al., J Intern Med. 237:135-41 (1995).

Fechheimer et al., (1987) Proc. Nat'l Acad. Sci. USA, 84,8463-8467.

Fraley et al., (1979) Proc Nat'l Acad. Sci. USA, 76,3348-3352.

Gaussin, V. et al. Proc. Natl. Acad. Sci. U.S. A. 99, 2878-2883 (2002).

Giet et al, J. Cell Biol.; 152,669-82 (2001).

Glaser, R., et al., J. Circulation 106, 17-19. (2002).

Goodell, M. A. et al. Nat. Med. 3, 1337-1345 (1997).

Gopal et al, Mol. Cell Biol., 5,1188-1190 (1985).

Graham and van der Eb, (1973) Virology, 52,456-467.

Hammond et al., (2001) Nat Rev Genet. 2,110-9.

Jackson, K. A. et al., J. Clin. Invest. 107, 1395-1402 (2001).

Jiang, Y. et al., Nature 418, 41-49. (2002).

Koh, G. Y., et al., (1995) J. Clin. Invest. 96, 2034-2042.

Kubasiak, L A, et al., Proc Natl Acad Sci USA. 99(20):12825-30 (2002).

Li et al., Ann Thorac Surg. 62(3):654-60; discussion 660-1 (1996).

Li et al., Circ Res. 78:283-8 (1996).

Li et al., J Thorac Cardiovasc Surg. 119(1):62-8 (2000).

Makino, S. et al., J. Clin. Invest. 103, 697-705 (1999).

Malouf, N. N. et al., Am J Pathol 158, 1929-1935. (2001).

Maniatis, et al., Molecular Cloning: A laboratory manual, Cold Spring Harbor Press, New York, 1982.

Martin et al., J. Cell Biol. 106:829-844 (1988).

McKinney-Freeman, S. L. et al., Proc Natl Acad Sci USA 99, 1341-1346. (2002).

Mishina, Y., et al., Genesis 32, 69-72. (2002).

Monzen, K. et al., Mol. Cell. Biol. 19, 7096-7105 (1999).

Murohara, T. et al., J Clin Invest 105, 1527-1536. (2000).

Nicolau and Sene, (1982) Biochim. Biophys. Acta, 721,185-190.

Nossuli, T. O. et al., Am. J. Physiol. Heart Circ. Physiol. 278, H1049-1055 (2000).

Oh, H. et al., Proc. Natl. Acad. Sci. U.S. A. 98, 10308-10313 (2001).

Orlic, D. et al., Nature 410, 701-705 (2001).

Orlic, D. et al., Proc. Natl. Acad. Sci. U.S. A. 98, 10344-10349. (2001).

Potter et al., (1984) Proc. Nat'l Acad. Sci. USA, 81,7161-7165.

Reinecke, H., et al., J. Mol. Cell. Cardiol. 34, 241-249 (2002).

Reinlib, L. & Field, L. Circulation 101, 182-187 (2000).

Rippe et al., Mol Cell Biol. 10(2):689-95 (1990).

Scorsin et al., Circulation 94(9 Suppl):II337-40 (1996).

Scorsin et al., J Thorac Cardiovasc Surg. 119:1169-75 (2000).

Soonpaa et al., Science. 264:98-101 (1994).

Soonpaa, M. H., et al., Am. J. Physiol. 271, H2183-H2189 (1996).

Soriano, P. Nat. Genet. 21, 70-71 (1999).

Stanisic et al., Invest. Urol. 16:19-22 (1978).

Stein et al., Biochem Biophys Res Commun. 291:1119-22 (2002).

Svoboda et al., Biochem Biophys Res Commun. 287(5):1099-104 (2001).

Svoboda et al., Development. 127(19):4147-56 (2000).

Tamura, H. et al., Exp Hematol 30, 957. (2002).

Tavazzi et al., Eur Heart J. 19 Suppl L:L33-5 (1998).

Terada, N. et al., Nature 416, 542-545. (2002).

Toma, C., Circulation 105, 93-98. (2002).

Tomita et al., Circulation. 100(19 Suppl):II247-56 (1999).

Tur-Kaspa et al., Mol. Cell Biol., 6,716-718 (1986).

Wu and Wu, Biochem., 27,887-892, (1988).

Wu and Wu, J. Biol. Chem., 262,4429-4432, (1987).

Yamashita, J. et al., Nature 408, 92-96 (2000).

Yang et al., Proc. Nat'l Acad. Sci. USA, 87,9568-9572, (1990).

Zhou, Y. Y. et al., Am J Physiol Heart Circ Physiol 279, H429-436. (2000).

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An isolated mammalian cardiomyocyte stem cell having c-kit^{neg}/CD31⁺/CD38⁺ and expressing telomerase reverse transcriptase.

2. The cell of claim 1, wherein the cell is CD45^{neg} and CD34^{neg}.

3. The cell of claim 1, wherein the cell is derived from bone marrow, umbilical cord blood, umbilical tissue, left atrial appendage, cardiac tissue, circulating endothelial progenitor cells, cardiac fibroblasts, adipose tissue or skin tissue

4. The cell of claim 1, wherein said cell exhibits spontaneous cell beating.

5. The cell of claim 1, wherein the cell expresses an adhesion protein.

6. The cell of claim 5, wherein the adhesion protein is selected from the group consisting of annexin A1 (Anxa1), nephronectin (Npnt), nidogen 2 (Nid2), pentaxin 3 (Ptx3), transmembrane 4 superfamily member 6 (Tm4sf6), and vascular cell adhesion molecule 1 (Vcam1).

7. The cell of claim 1, wherein the cell expresses macrophage colony stimulating factor 1.

8. The cell of claim 1, wherein the cell expresses a receptor.

9. The cell of claim 8, wherein the receptor is selected from the group consisting of fibroblast growth factor receptor 1 (Fgfr1), cytokine receptor-like factor 1 (Crlf1), interleukin 4 receptor alpha (Il4ra), platelet derived growth factor receptor alpha polypeptide (Pdgfra), and tumor necrosis factor receptor superfamily member 6 (Tnfrsf6).

10. The cell of claim 1, wherein the cell is capable of differentiating into cardiac muscle.

11. The cell of claim 1, wherein the cell is capable of differentiating into vascular cells.

12. A pharmaceutical composition comprising a therapeutically effective amount of isolated stem cells of claim 1 and a pharmaceutical acceptable carrier.

13. The composition of claim 12 further comprising a transcription factor for cardiac development.

14. The composition of claim 13, wherein the transcription factor is Nkx2.5.

15. The composition of claim 12 further comprising a factor that enhances the activity of the Wnt/β-catenin signaling pathway.

16. The composition of claim 12 further comprising a factor that enhances the activity of BMP (bone morphogenic protein).

17. A kit comprising a pharmaceutical composition of claim 12 to treat a cardiovascular disease.

18. An isolated c-kit negative cardiac derived stem cell.

19. The cell of claim 18, wherein the cell expresses CD31/PECAM-1 or CD38.

20. The cell of claim 18, wherein the cell expresses an adhesion protein.

21. The cell of claim 20, wherein the adhesion protein is selected from the group consisting of annexin A1 (Anxa1), nephronectin (Npnt), nidogen 2 (Nid2), pentaxin 3 (Ptx3), transmembrane 4 superfamily member 6 (Tm4sf6), and vascular cell adhesion molecule 1 (Vcam1).

22. The cell of claim 18, wherein the cell expresses macrophage colony stimulating factor 1.

23. The cell of claim 18, wherein the cell expresses a receptor.

24. The cell of claim 23, wherein the receptor is selected from the group consisting of fibroblast growth factor receptor 1 (Fgfr1), cytokine receptor-like factor 1 (Crlf1), interleukin 4 receptor alpha (Il4ra), platelet derived growth factor receptor alpha polypeptide (Pdgfra), and tumor necrosis factor receptor superfamily member 6 (Tnfrsf6).

25. The cell of claim 18, wherein the cell expresses telomerase reverse transcriptase.

26. The cell of claim 18, wherein the cell is isolated from a non-myocyte fraction.

27. The cell of claim 18, wherein the cell is capable of differentiating into cardiac muscle.

28. The cell of claim 18, wherein the cell is capable of differentiating into vascular cells.

29. The cell of claim 18, wherein said cell exhibits spontaneous cell beating.

30. A pharmaceutical composition comprising a therapeutically effective amount of isolated stem cells of claim 18 and a pharmaceutical acceptable carrier.

31. The composition of claim 30 further comprising a transcription factor for cardiac development.

32. The composition of claim 31, wherein the transcription factor is Nkx2.5.

33. The composition of claim 30 further comprising a factor that enhances the activity of the Wnt/β-catenin signaling pathway.

34. The composition of claim 30 further comprising a factor that enhances the activity of BMP (bone morphogenic protein).

35. A kit comprising a pharmaceutical composition of claim 30 to treat a cardiovascular disease.

36. A method of treating a subject suffering from a cardiovascular disease comprising the step of administering to the subject cardiac stem cells, wherein the stem cells are c-kit^{neg}/CD3⁺/CD38⁺ and express telomerase reverse transcriptase.

37. The method of claim 36, wherein the cardiovascular disease is selected from the group consisting of coronary artery disease, myocardial infarction, ischemic heart disease and heart failure.

38. The method of claim 36, wherein the cells differentiate into at least one cardiac cell type selected from the group consisting of myocytes, endothelial cells, vascular smooth muscle cells, and fibroblasts.

39. The method of claim 36, wherein the cells are admixed in a pharmaceutical acceptable carrier.

40. The method of claim 36, wherein administering is via a parenteral route.

41. The method of claim 40, wherein the parenteral route is intravenously.

42. The method of claim 36, wherein administering is via direct injection into the heart of the subject.

43. The method of claim 36, wherein the cells are autologous, heterologous, or homologous.

44. The method of claim 36, wherein administering is via implantation of the cells that are comprised on a matrix.

45. A method of treating a subject suffering from an infarcted myocardium comprising the step of administering to the subject an effective amount of cardiac stem cells having c-kit^{neg}/CD31⁺/CD38⁺ and expressing telomerase reverse transcriptase, wherein the amount repairs the infarcted myocardium.

46. The method of claim 45, wherein the repairs comprise regeneration of cardiomyocytes.

47. A method of targeting injured myocardium comprising the step of administering to the subject cardiac stem cells having c-kit^{neg}/CD31⁺/CD38⁺ and expressing telomerase reverse transcriptase, wherein the cells migrate and attach to the injured myocardium.

48. The method of claim 47, wherein the cells differentiate into at least one cardiac cell type selected from the group consisting of myocytes, smooth muscle cells and endothelial cells.

49. A method of repairing an injured myocardium comprising the step of administering to a subject an effective amount cardiac stem cells having c-kit^{neg}/CD31⁺/CD38⁺ and expressing telomerase reverse transcriptase, wherein the amount is effective in repairing the injured myocardium.

50. The method of claim 49, wherein repairing comprises at least partially restoring structural integrity to the injured myocardium.

51. The method of claim 49, wherein repairing comprises at least partially restoring functional integrity to the injured myocardium.

52. A method of repairing injured coronary vessels comprising the step of administering to a subject an effective amount of cardiac stem cells having c-kit^{neg}/CD31⁺/CD38⁺ and expressing telomerase reverse transcriptase, wherein the amount is effective in regenerating vascular cells to repair the vessels.

53. A method of generating myocytes comprising the steps of: obtaining cardiac stem cells having c-kit^{neg}/CD31⁺/CD38⁺ and expressing telomerase reverse transcriptase; and differentiating the stem cells to generate myocytes, wherein differentiating is performed in vitro.

54. The method of claim 53, wherein differentiating further comprises the addition of a transcription factor for cardiac development.

55. The method of claim 54, wherein the cardiac transcription factor is Nkx2.5.

56. A method of treating damaged myocardium in a subject comprising the steps of:
obtaining autologous cardiac stem cells having c-kit^{neg}/CD31⁺/CD38⁺ and expressing telomerase reverse transcriptase from the subject; proliferating the stem cells in vitro; and administering intravenously to the subject the stem cells, wherein the stem cells migrate to the damaged myocardium.

57. The method of claim 56, wherein obtaining comprises performing a tissue biopsy.

58. A method of treating heart failure in a subject comprising the step of administering to the subject an effective amount cardiac stem cells that are c-kit^{neg}/CD31⁺/CD38⁺ and express telomerase reverse transcriptase, wherein the amount is effective in at least partially restoring cardiac function.

59. The method of claim 58, wherein the heart failure comprise the loss of cardiomyocytes.

60. The method of claim 59, wherein the loss of cardiomyocytes is caused by apoptosis.

61. A method of modulating the loss of cardiomyocytes in a subject comprising the step of administering to the subject an effective amount cardiac stem cells that are c-kit^{neg}/CD31⁺/CD38⁺ and express telomerase reverse transcriptase, wherein the amount is effective in at least partially restoring cardiomyocytes.

62. The method of claim 61, wherein the loss of cardiomyocytes is caused by apoptosis.


SEQUENCE LISTING (8 sequences)

SEQ ID NO: 1 - DNA, 20 bp, Artificial Sequence
  Primer
ggaagaggtgcagtgcgatg

SEQ ID NO: 2 - DNA, 20 bp, Artificial Sequence
  Primer
acctggaacacttctcagct

SEQ ID NO: 3 - DNA, 20 bp, Artificial Sequence
  Primer
ggaagaggtgcagtgcgatg

SEQ ID NO: 4 - DNA, 20 bp, Artificial Sequence
  Primer
acctggaacacttctcagct

SEQ ID NO: 5 - DNA, 20 bp, Artificial Sequence
  Primer
ggaagaggtgcagtgcgatg

SEQ ID NO: 6 - DNA, 20 bp, Artificial Sequence
  Primer
acctggaacacttctcagct

SEQ ID NO: 7 - DNA, 30 bp, Artificial Sequence
  Primer
ccttttcacggtttctttacacacacactg

SEQ ID NO: 8 - DNA, 30 bp, Artificial Sequence
  Primer
ggtcagctaatactcatagcaaattcagcc